(19) United States
(12) Patent Application Publication
(10) Pub. No.: US 2016/0076026 A1
(43) Pub. Date: Mar. 17, 2016

(54) DIAGNOSTIC AND THERAPEUTIC METHODS RELATING TO MICRORNA-144

(71) Applicant: THE HOSPITAL FOR SICK CHILDREN, Toronto (CA)

(72) Inventors: Andrew Redington, Cincinnati, OH (US); Jing Li, Toronto (CA)

(21) Appl. No.: 14/776,970

(22) PCT Filed: Mar. 14, 2014
(86) PCT No.: PCT/IB2014/001175

§ 371 Date: Sep. 15, 2015

Related U.S. Application Data
(60) Provisional application No. 61/800,738, filed on Mar. 15, 2013.
(60) Provisional application No. 61/889,489, filed on Oct. 10, 2013.

(51) Int. Cl. C12N 15/113; A61K 31/7105; A61K 45/06

(52) U.S. Cl. CPC C12N 15/113; A61K 45/06; A61K 31/7105; C12N 2310/141; C12N 2310/3515

(57) ABSTRACT

The invention provides methods based on the use of miRNA-144 as a predictive factor (e.g., as a companion diagnostic) and/or as a prophylactic or therapeutic agent.

### RELATED APPLICATIONS

This application claims the benefit under 35 U.S.C. §119(e) of U.S. Provisional Application Ser. No. 61/800,738 entitled “DIAGNOSTIC AND THERAPEUTIC METHODS RELATING TO MICRORNA-144” and filed on Mar. 15, 2013, and U.S. Provisional Application Ser. No. 61/889,489, entitled “DIAGNOSTIC AND THERAPEUTIC METHODS RELATING TO MICRORNA-144” filed on Oct. 10, 2013, the entire contents of both of which are incorporated herein by reference.

### FIELD OF INVENTION

The invention provides methods and compositions for detection and use of particular microRNAs, including as biomarkers and as companion diagnostics.

### BACKGROUND OF INVENTION

Remote ischemic conditioning (RIC) induced by transient limb ischemia has been shown to invoke potent myocardial protection in multiple animal models, and has rapidly translated to positive clinical trials. The mechanism by which remote ischemic conditioning provides cardioprotective effect is incompletely understood. Nevertheless, it has been shown that remote ischemic conditioning induces the release of cardioprotective factor(s) in the blood and that such factor(s) can be transferred across individuals and across species. Previous studies characterized the cardioprotective factor(s) as a dialyzable (less than 15 kDa), hydrophobic factor(s). The release of such factors can be induced by other RIC-like interventions such as direct femoral nerve stimulation, nociceptive c-fiber stimulation via topical capsaicin, transcutaneous electrical nerve stimulation (TENS), intra-arterial adenosine and local adenosine release.

### SUMMARY OF INVENTION

The invention is based, in part, on the surprising discovery that the level of miRNA-144 in a subject can be used to (1) predict whether the subject is likely to be responsive to local or remote ischemic conditioning (IC) (or other IC-like interventions) and/or (2) monitor the efficacy of ischemic conditioning (or other IC-like intervention) on a subject. The invention therefore contemplates measuring miRNA-144 levels in a subject, in some instances prior to IC or IC-like intervention, in order to identify the subject as either likely or unlikely to be responsive to IC or IC-like intervention. Certain subjects may then undergo IC or IC-like intervention while others may not depending on their miRNA-144 level.

The invention alternatively or additionally contemplates measuring miRNA-144 levels in a subject that is undergoing or has undergone IC or an IC-like intervention in order to determine whether the IC or IC-like intervention is efficacious and/or to determine if and when further IC or an IC-like intervention is warranted. To this end, the invention contemplates that miRNA-144 can be used as a companion diagnostic for IC or an IC-like intervention.

The invention is also based, in part, on the additional surprising discovery that miRNA-144 mediates beneficial effects associated with IC such as remote IC. This is surprising, in part, because it had been previously reported that miRNA-451, and not miRNA-144, was responsible for the cardioprotective benefits associated with local IC. The invention, on the other hand, is based in part on the finding that miRNA-144 when administered to a subject can have the same beneficial effects as remote IC and that such beneficial effects are not observed when an antagonist of miRNA-144 is administered alongside miRNA-144 or alongside remote IC.

Thus, the invention further contemplates the prophylactic and/or therapeutic use of miRNA-144, alone or in combination with (1) IC or an IC-like intervention and/or (2) other secondary therapeutic agents, in subjects in need of IC-induced benefits. Such subjects include those at risk of experiencing, those experiencing, or those that have experienced an ischemic event such as a myocardial infarction or a stroke, those at risk of experiencing or those experiencing restenosis, those at risk of experiencing, those experiencing, or those that have experienced traumatic injury such as hemorrhagic shock, those generally in need of cardioprotection, as well as those seeking performance enhancement benefits. Such subjects also include those having or at risk of developing cancer, a neurodegenerative disorder, a gastrointestinal disorder, a metabolic disorder, a cardiovascular disorder, or a pulmonary disease. Such subjects also include those having an infection such as a bacterial infection, a viral infection, a fungal infection, a mycobacterial infection, or a parasitic infection.

Accordingly, the invention provides methods, compositions and devices relating to the use of miRNA-144 as a biomarker of efficacy of future, ongoing or past IC or IC-like intervention, and/or as a therapeutic agent or as a health promoting agent.

Thus, in one aspect, the invention provides a method comprising measuring a level of miRNA-144 in a biological sample from a subject, and identifying the subject as not likely to respond to ischemic conditioning (IC) or an IC-like intervention based on the level of miRNA-144 relative to a control.

In another aspect, the invention provides a method comprising measuring a level of miRNA-144 in a biological sample from a subject, and identifying the subject as likely to respond to ischemic conditioning (IC) or an IC-like intervention based on the level of miRNA-144 relative to a control. In some embodiments, the method further comprises performing IC or an IC-like intervention on the identified subject.

In another aspect, the invention provides a method comprising measuring a level of miRNA-144 in a biological sample from a subject, and identifying the subject as likely to respond to an miRNA-144 therapy based on the level of miRNA-144 relative to a control. In some embodiments, the method further comprises administering an miRNA-144 therapy to the identified subject.

In some embodiments, the level of miRNA-144 is measured before performing IC or an IC-like intervention on the subject or before administering an miRNA-144 therapy to the subject. In some embodiments, the level of miRNA-144 is measured after performing IC or an IC-like intervention on the subject or after administering an miRNA-144 therapy to the subject. In some embodiments, the level of miRNA-144 is measured before and after performing IC or an IC-like intervention on the subject or before and after administering an miRNA-144 therapy to the subject. In some embodiments, the level of miRNA-144 is measured once or twice.

In some embodiments, the control is a pre-determined level of miRNA-144. In some embodiments, the control is a time-course of miRNA-144 levels.

In some embodiments, the subject is at risk of having, or is experiencing, or has experienced an ischemic injury. In some embodiments, the subject is at risk of having, or is experiencing, or has experienced a myocardial infarction, a stroke, restenosis, or traumatic injury. In some embodiments, the subject is at risk of developing, or has developed a condition selected from diabetes, metabolic syndrome, cancer, Crohn's disease, ulcerative colitis, pulmonary disease, atherosclerosis, or cardiomyopathy. In some embodiments, the subject is scheduled to have surgery. In some embodiments, the surgery is cardiac surgery or cardiovascular surgery. In some embodiments, the subject is a healthy subject and IC or IC-like intervention or miRNA-144 administration is performed to enhance performance. In some embodiments, the subject is human.

In another aspect, the invention provides a method comprising administering to a subject in need thereof an miRNA-144 therapy in an effective amount.

In some embodiments, the subject in need thereof is a subject at risk of experiencing, or a subject experiencing, or a subject that has experienced an ischemic injury. In some embodiments, the ischemic injury is a myocardial infarction, a stroke, restenosis, or traumatic injury. In some embodiments, the subject is in need of cardioprotection and the miRNA-144 is administered before, during and/or following an ischemic event that causes cardiac ischemia. In some embodiments, the subject is not in need of cardioprotection. In some embodiments, the subject is not at risk of experiencing, or is not experiencing, or has not experienced a myocardial infarction or other event that causes cardiac ischemic injury. In some embodiments, the ischemic injury is surgery. In some embodiments, the surgery is cardiac surgery or cardiovascular surgery.

In some embodiments, IC or an IC-like interventions has been, is being, and/or will be performed on the subject. In some embodiments, the IC-like intervention is transcutaneous electrical nerve stimulation.

In some embodiments, the subject is human.

In some embodiments, the method further comprises administering to the subject an additional therapeutic agent. In some embodiments, the method further comprises administering to the subject an angiotensin-converting enzyme (ACE) inhibitor. In some embodiments, the method further comprises administering to the subject an angiotensin II receptor blocker. In some embodiments, the method further comprises administering to the subject an anti-platelet therapy.

In some embodiments, the subject is having or likely to experience restenosis following a medical intervention. In some embodiments, the medical intervention is an intravascular stent placement, angioplasty or non-vascular stent placement. In some embodiments, the intravascular stent placement is an arterial stent placement, a venous stent placement, a bare-metal stent placement, or a drug-eluting stent placement. In some embodiments, the medical intervention is a esophageal stent placement, a tracheal stent placement, a ureteral stent placement, or a bile duct stent placement.

In some embodiments, the miRNA-144 therapy comprises a nucleic acid consisting of a nucleotide sequence of SEQ ID NO:1, 2, 4 or 6. In some embodiments, the miRNA-144 therapy comprises nucleic acid comprising a nucleotide sequence that is complementary to SEQ ID NOs:1, 2, 4 or 6. In some embodiments, the nucleic acid comprises one or more non-naturally occurring backbone linkage. In some embodiments, the nucleic acid comprises one or more non-naturally occurring nucleotide or nucleotide analogs or nucleotide modifications. In some embodiments, the nucleic acid comprises a cholesterol modified nucleotide or nucleotide analog.

In another aspect, the invention provides a method for enhancing physical performance comprising administering an miRNA-144 therapy to a subject having a cardiovascular condition prior to a physical activity in order to enhance performance of the physical activity by the subject.

In another aspect, the invention provides a method for enhancing physical performance comprising administering an miRNA-144 therapy to a healthy subject prior to a maximal physical activity.

In some embodiments, the miRNA-144 therapy is administered within 24 hours, within 2 hours, or within 20 minutes prior to the physical activity.

In some embodiments, the subject is human.

In some embodiments, the method causes about a 1.5% improvement in maximal physical activity.

In some embodiments, the miRNA-144 therapy comprises a nucleic acid consisting of a nucleotide sequence of SEQ ID NO:1, 2, 4 or 6. In some embodiments, the miRNA-144 therapy comprises nucleic acid comprising a nucleotide sequence that is complementary to SEQ ID NOs:1, 2, 4 or 6. In some embodiments, the nucleic acid comprises one or more non-naturally occurring backbone linkage. In some embodiments, the nucleic acid comprises one or more non-naturally occurring nucleotide or nucleotide analogs or nucleotide modifications. In some embodiments, the nucleic acid comprises a cholesterol modified nucleotide or nucleotide analog.

It is to be understood that the invention also contemplates the use of miR-144 precursor as a biomarker and as a therapeutic in a manner similar to that contemplated for mature miR-144. This disclosure describes the invention in the context of miR-144; however this is to be understood to be for the sake of brevity only and not to exclude the use of miR-144 precursor for the recited aspects and embodiments of the invention.

The present invention further encompasses methods of making and/or using one or more of the embodiments described herein.

Other advantages and novel features of the present invention will become apparent from the following detailed description of various non-limiting embodiments of the invention when considered in conjunction with the accompanying Figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF DRAWINGS

FIG. 1A is a graph showing the validation of miRNA microarray data. The y-axis is fold expression of miRNA compared to control. The levels of miR-27a, miR-144 and miR-489 are shown in both control (first bar in each pairing) and after rIPC (second bar in each pairing). The decrease in miR-27 level after rIPC was significant at the level of p<0.01. The increase in miR-144 level after rIPC was significant at the level of p<0.05. The increase in miR-489 level after rIPC was significant at the level of p=0.09.

FIG. 1B shows that myocardial miR-144 levels significantly decrease compared to controls after ischemia-reperfusion injury (n=4). Data are shown as mean±SEM. Statistical significance is shown as *p<0.05, §P<0.01 vs. Control.

FIG. 2 is a graph showing the effect of systemic administration of antagomir-144 and miR-144 on cardiac miRNA-144 level. The bars represent from left to right: PBS, antagomir-Co, antagomir-144, and mir-144. The last two bars have a significance level of p=0.06 and p<0.05 respectively.

FIG. 3 is a graph showing the effect of RIC, antagomir-144, RIC and antagomir-144, and miRNA-144 on infarct sizes. The bars represent from left to right: PBS, PBS+rIPC, mir-Co+rIPC, antagomir-144+rIPC, antagomir-144 and mir-144. * indicates p<0.05 vs. PBS. § indicates p=0.01 vs. PBS+rIPC.

FIG. 4 is a graph showing the effect of RIC, antagomir-144, RIC and antagomir-144, and miRNA-144 on LVEDP after experimentally induced infarcts.

FIG. 5 is a graph showing the effect of RIC, antagomir-144, RIC and antagomir-144, and miRNA-144 on LVDP after experimentally induced infarcts.

FIG. 6 is a graph showing the effect of RIC on miRNA-144 plasma level.

FIG. 7 shows that myocardial miR-144 level after systemic administration of miR-144. (A) Myocardial miR-144 level (fold vs. PBS) after 50 min injection, and 1 day after three days of miR-144 intravenous administration. miR-144 levels were increased over two-fold, compared to PBS control. Bars represent from left to right: PBS, miR-Co, miR-144 Day 1, and miR-144 Day 3. (B) Representative Western blot and quantification of phosphorylated-Akt (P-AKT, ser 473) protein expression in the myocardium (fold vs. PBS) 1 hour after miR-144 injection. P-Akt was unchanged in PBS and miR-Co groups, but showed a 2-fold increase after miR-144 injection. Bars represent from left to right: PBS, miR-Co, and miR-144. (C and D) Mouse cardiac miR-144 levels (fold vs. PBS) after IR injury was higher in mouse heart both by pretreatment with intravenous miR-144 (C) and remote IC (D). Bars in (C) represent from left to right: PBS, miR-Co, and miR-144 Day 1. Bars in (D) represent from left to right: sham and rIPC. Statistical significance is shown as *p<0.05 vs. PBS. P=0.08 in (D).

FIG. 8 shows that intravenous miR-144 provides early and delayed cardioprotection. (A and C) The recovery of left ventricular developed pressure (LVDP) was improved in miR-144 Day 1 and miR-144 Day 3 groups. (B and D) Diastolic recovery (LVEDP=left ventricular end-diastolic pressure). * denotes a statistically significant difference between miR-144 Day 1 and miR-144 Day 3 groups vs. PBS (* p<0.05) after 60 minutes of reperfusion. (A and B): circles are PBS Day 1, pink triangles are miR-Co, blue triangles are miR-144 Day 1. (C and D): circles are PBS Day 3, triangles are miR-144 Day 3. (E and F) Myocardial infarct size (%) was measured by TTC staining. A representative basal left ventricular section is presented for each group. Pre-treatment with intravenous scrambled miR control had no effect (p=ns compared to PBS), whereas there was significant cardioprotection 50 minutes after a single injection, and 1 day after 3 daily injections of miR144. Bars in (E) represent from left to right: PBS, miR-Co, and miR-144 Day 1. Bars in (F) represent from left to right: PBS and miR-144 Day 3. Statistical significance is shown as *p<0.05 vs. PBS.

FIG. 9 shows the effects of remote IC on circulating human and mouse miR-144 levels. Plasma was collected from mice and human to measure miR-144 levels using MicroRNA Stem-Loop RT-PCR. (A) Plasma miR-144 levels (fold change) in mice subjected to remote IC (4×5 minute cycles of limb ischemia/5 minutes reperfusion). There was a 2-fold increase in circulating miR-144 levels. Bars represent from left to right: Control and rIPC. (B) Circulating miR-144 levels (fold change) before and after remote IC in eight human volunteers. Remote IC was administered using a blood pressure cuff around the upper arm. Blood was collected before and after remote IC. There was a 1.6 fold increase in miR-144 levels following remote IC. Bars represent from left to right: pre-rIPC and post-rIPC. Statistical significance is shown as *p<0.05 vs. Control, §p<0.01 vs. pre-remote IC.

FIG. 10. Exosomes were isolated from serum of remote IC-treated and control animals using ExoQuick precipitation solution. The exosome extract was diluted 1:20 for analysis with the NanoSight. Data collection was performed using NanoSight software (V. 2.3) with the detection threshold set at 6 to maximize sensitivity while minimizing noise. Duplicate measurements were made for each sample. Overall there was no difference in the absolute numbers of circulating exosomes following remote IC. The inset panel shows a representative EM image of the exosome sample, and western blotting shows positive binding with the exosomal membrane marker CD63. The y-axis represents exosome concentration (E6 particles/ml). The x-axis represents exosome size (nm).

FIG. 11. The levels of miR-144 in circulating serum exosomes. Exosomes were isolated from mouse serum using ExoQuick. (A) miR-144 level in mouse serum exosomes (fold change) was measured using Stem loop RT-PCR. The bars represent from left to right: control and rIPC. (B) Precursor miR-144 levels in serum exosomes (compared to control) was determined by miScript Precursor Assay. The bars represent from left to right: control and rIPC. (C) Following exosome isolation, miR-144 levels were measured in the mouse exosome-poor supernatant. miR-144 levels (fold change) were significantly increased in exosome-poor supernatant after remote IC. The bars represent from left to right: control and rIPC. (D) To elucidate a potential extracellular miRNA transport mechanism, the binding of miR-144 to Ago2 protein in blood serum by subjecting anti-Ago2 immunoprecipitates to TaqMan miRNA assay was performed. Ago2-bound miR-144 levels was increased following remote IC. y-axis is miR-144 level in Ago2 IPs (fold change). The bars represent from left to right: control and rIPC. Statistical significance is shown as *p<0.05 vs. Control.

Non-limiting embodiments of the present invention will be described by way of example with reference to the accompanying Figures, which are schematic and are not intended to be drawn to scale. For purposes of clarity, not every component is labeled in every figure, nor is every component of each embodiment of the invention shown where illustration is not necessary to allow those of ordinary skill in the art to understand the invention.

### DETAILED DESCRIPTION OF INVENTION

The invention is based, in part, on the surprising finding that miRNA-144 levels in a subject can be used to determine whether ischemic conditioning (IC) or an IC-like intervention has been or is likely to be efficacious in the subject. The invention contemplates measuring miRNA-144 levels in a subject prior to performing IC or an IC-like intervention on the subject in order to determine whether the subject is likely to be responsive to IC or IC-like intervention. Subjects identified as likely to respond, based on their miRNA-144 levels, may then undergo IC or IC-like intervention. Subjects identified as unlikely to respond, based on their miRNA-144 levels, may not undergo IC or IC-like intervention and may instead be treated using a different modality or therapy. The invention therefore prevents the use of IC or IC-like intervention in a subject who will likely receive no benefit therefrom.

The invention further contemplates measuring miRNA-144 levels in a subject after IC or an IC-like intervention has been performed to determine whether the subject has (or has not) responded to the IC or IC-like intervention. Identifying subjects as non-responsive to IC or an IC-like intervention prevents treating such subjects in a similar manner again. Conversely, partial responders may benefit from further treatment by IC, and such treatment could be guided by miRNA-144 levels.

The invention still further contemplates determining the timing of further IC interventions based on miRNA-144 levels in a subject. Decreasing levels of miRNA-144 in a subject indicate, in some instances, that further IC or IC-like intervention is warranted. High steady-state levels of miRNA-144 or increasing levels of miRNA-144 indicate, in some instances, that further IC or IC-like interventions are not warranted at least in the short term. In this way, the invention provides a method for determining optimal times for performing IC or an IC-like intervention on a subject for maximal benefit, and thereby avoids performing IC or an IC-like intervention at a time when a subject is unlikely or less likely to benefit therefrom.

Thus, it is to be understood that, in some aspects of the invention, miRNA-144 levels are intended as a marker of the amount of IC treatment a subject would benefit from (dose-response), and of whether a subject is likely to respond at all or at a particular time to IC or IC-like intervention. In this regard, miRNA-144 is considered to be a companion diagnostic.

The invention is based, also in part, on the additional surprising finding that miRNA-144 is a mediator of remote IC. As shown in the Examples, miRNA-144 provides cardioprotection at a level similar to that of remote IC. Moreover, the Examples also show that the cardioprotective effects of remote IC are reduced or eliminated upon administration of a miRNA-144 antagonist. These findings are particularly surprising because it had been previously reported that miRNA-144 was not a mediator of local IC induced cardioprotection and that rather another miRNA on the same genomic cluster as miRNA-144 (i.e., miRNA-451) was actually the mediator of local IC protection.

Thus, the invention further contemplates use of miRNA as a prophylactic and/or therapeutic agent itself in place of or alongside IC or an IC-like intervention (and/or other secondary therapeutic agent) in subject in need thereof. miRNA-144 can therefore be used to predict likelihood of response to its own use as a prophylactic and/or therapeutic.

Some of the methods provided herein are theranostic methods that involve the selection of a subject for treatment with IC, an IC-like invention, or miRNA-144 itself. Such theranostic methods help to avoid performing a procedure or administering a medicament to subjects that will likely derive no benefit therefrom. Such methods may comprise a step of performing a IC or an IC-like intervention or administering miRNA-144 to a subject identified as likely to respond.

As described in greater detail herein, in accordance with the invention, microarray studies have established that IC including rIC increases and ischemic-reperfusion (I-R) injury decreases miR-144 levels in mouse myocardium, with the latter being rescued by both IC (including rIC) and intravenous administration of miR-144. It has also been shown in accordance with the invention that systemic treatment with miR-144 resulted in increased levels of phosphorylated AKT and induced early and delayed cardioprotection with improved functional recovery and reduction in infarct size similar to that achieved by IC. Conversely, systemic administration of a specific antisense oligonucleotide reduced myocardial levels of miR-144 and abrogated cardioprotection by IC. It has been further shown that IC increases plasma miR-144 levels in mice and humans. No change in plasma microparticle (50-400 nM) numbers or their miR-144 content was observed; however, there was an almost 4-fold increase in miR-144 precursor in the exosome pellet, and a significant increase in miR-144 levels in exosome-poor serum which, in turn, was associated with increased levels of the miR carriage protein Argonaute-2. These results indicate that miR-144 plays a pivotal role in cardioprotection.

miRNA-144 Generally

miRNAs are short, non-coding RNAs of about 18 to about 25 nucleotides in length. miRNAs act as repressors of target mRNAs by enhancing their degradation or inhibiting translation therefrom. The degree of the miRNA effect depends on the degree of its complementarity with its target mRNA.

The sequences of numerous miRNA are known and publicly available. Synthesis of miRNA (e.g., for prophylactic or therapeutic purposes) and miRNA-specific probes (e.g., for diagnostic purposes) is within the ordinary skill in the art based on this information. miRNA nucleotide sequences can be accessed at for example the website of the miRNA Registry of the Sanger Institute (Wellcome Trust), or the website of Ambion, Inc.

The nucleotide sequence of miRNA-144 precursor form is 5′UGGGGCCCUGGCUGGGAUAUCAUCAUAUACUGUAAGUUUGCGAUGAGACA CUACAGUAUAGAUGAUGUACUAGUCCGGGCACCCCC 3′ (SEQ ID NO:1). This precursor form adopts a hairpin structure from which is excised the mature miRNA sequence through the activity of Dicer.

The nucleotide sequence of human miRNA-144 3′ mature form is 5′ UACAGUAUAGAUGAUGUACU 3′ (SEQ ID NO: 2, 20 nucleotides).

The invention contemplates the use of nucleic acids consisting of or comprising the mature miRNA-144 sequence or the precursor miRNA-144 sequence.

In some embodiments, the invention contemplates use of nucleic acid comprising SEQ ID NO:2 and additional flanking nucleotides on either or both the 5′ and 3′ ends of this nucleotide sequence. An example of such a nucleic acid is used in some of the Examples and has the nucleotide sequence of 5′ UACAGUAUAGAUGAUGUACUAG 3′ (SEQ ID NO:6). This sequence has two additional nucleotides at the 3′ end of the miRNA mature sequence provided as SEQ ID NO:2. The number of flanking nucleotides may be 1, 2, 3, 4, 5, 6, 7, 8, 9, 10, or more on either or both sides. The number and sequence of nucleotides added to the 5′ and 3′ ends may be the same or it may be different. The invention embraces such nucleic acids to the extent that they can be used in the methods of the invention in the same manner as mature or precursor form of miRNA-144 can be used, including for example as probes, as in vitro controls, as therapeutic agents that induce IC-like effects, and the like. In some instances, such nucleic acids may have the same or nearly the same activity as mature or precursor form miRNA-144 (e.g., +/−10% or +/−5% or +/−1% of mature or precursor form miRNA-144). In some instances, such nucleic acids have lower activity than mature or precursor form miRNA-144 but are still considered useful in one or more methods of the invention. Lower activity may be at least about 30%, about 40%, about 50%, about 60%, about 70%, about 80%, or about 90% of the activity of mature or precursor form miRNA-144. The activity will generally be above background or negative control activity. In some instances, such nucleic acids may have higher activity than mature of precursor form miRNA-144. Higher activity may be at least about 110%, about 120%, about 130%, about 140%, about 150%, about 160%, about 170%, about 180%, about 190%, about 200%, or more of the activity of mature of precursor form miRNA-144.

In some embodiments such as the prophylactic and/or therapeutic methods of the invention, the nucleic acids do not comprise the miRNA-451 nucleotide sequence (or its complete complementary sequence). The nucleotide sequence of human mature miRNA-451 is AAACCGUUACCAUUACUGAGUUU (SEQ ID NO: 7).

The miRNA-144 nucleic acids of the invention may range in length from about 20 to about 100 nucleotides, or about 20 to about 50 nucleotides, or about 20 nucleotides to about 30 nucleotides. In some embodiments, they may be more than 100 nucleotides, including for example if they are presented in a vector such as a virus or virus like construct for in vivo production.

In some embodiments, the miRNA-144 nucleic acids of the invention are isolated. This means that the miRNA-144 is physically separated from its natural environment which may be genomic DNA, or a cell, or a cell lysate, or an in vitro chemical reaction mixture.

miRNA-144 as a Companion Diagnostic

The detection or diagnostic methods of the invention involve measuring miRNA-144 levels in a sample taken from a subject and comparing those levels to a control or a set of controls in order to determine if the subject is likely to respond to IC or IC-like intervention or miRNA-144 therapy itself. The miRNA-144 level at a given time point may be used in some instances. The change in miRNA-144 level between two time points may be used in other instances. Accordingly, the invention contemplates measuring miRNA-144 levels in a subject one or more times, including comparing the miRNA-144 levels between subsequent time points. miRNA-144 levels that are increasing may indicate that IC or an IC-like intervention or miRNA-144 therapy may be delayed. miRNA-144 levels that are decreasing may indicate that IC or IC-like intervention or miRNA-144 therapy may be performed without delay.

Thus, in some embodiments, the control(s) are levels of miRNA-144 taken from one or more subjects known to be responsive to IC or an IC-like intervention and previously subjected to IC or IC-like intervention. The control levels may be measured in such control subjects before IC (pre-IC levels) and/or after IC (post-IC levels). In some embodiments, the control is actually a time-course of miRNA-144 levels including levels before IC, through IC, and following IC (or IC-like intervention). The time-course may provide steady-state pre-IC levels, followed by an increasing levels as a result of IC, followed by decreasing levels as the IC-induced effects and benefits start to wane and the subject returns to a lower steady state.

Accordingly, a measured miRNA-144 level in a subject can be mapped against the established response curve and the likelihood of response in the subject can be determined.

The control levels and time-course may be established prior to analysis of any given subject. The control levels and time-course may be continually updated with data from additional subjects. It is contemplated that each control levels and/or each data point on the time-course may be a range of values to reflect the variability between subjects. The methods provided herein do not require that a control level be measured every time a subject is tested. Rather, it is contemplated that control levels of miRNA-144 are obtained and recorded and that any test level is compared thereto. Such pre-determined control levels (or ranges) may also be referred to herein as pre-determined threshold levels (or ranges).

Subjects having miRNA-144 levels above a particular threshold may not be treated with IC or IC-like intervention or miRNA-144 at all or at that particular time. Subjects having miRNA-144 levels below a particular threshold may be treated with IC or IC-like intervention or miRNA-144.

Subjects having increasing miRNA-144 levels may not be treated with IC or IC-like intervention or miRNA-144 at that particular time. IC or IC-like intervention in such subjects may be delayed until their miRNA-144 levels begin falling or are below a particular threshold.

Subjects having decreasing miRNA-144 levels may be treated with IC or IC-like intervention or miRNA-144 at that time or at any time thereafter. Alternatively, IC or IC-like intervention in such subjects may be delayed until their miRNA-144 levels are below a particular threshold.

Some methods may utilize a control that is a maximum miRNA-144 level (or range) observed in subjects in response to IC or IC-like intervention. miRNA-144 levels that are 50% or more, or 60% or more, or 70% or more, or 80% or more, or 90% or more, or 95% or more or 100% of such control level (or range) may indicate that a subject need not undergo IC or an IC-like intervention or miRNA-144 therapy at that time, particularly if the subject is experiencing an increase in the level of miRNA-144 at the time.

In some embodiments, miRNA-144 levels are lower than a predetermined blood level. The predetermined blood level may be established by measuring the dose-response curve to IC in subjects that are responsive to such intervention. Thus, with the knowledge provided herein, one of ordinary skill in the art can generate dose-response curves that correlate miRNA-144 levels with IC intervention. Such dose-response curves may take the form of the time-course plots discussed herein.

In some embodiments, the invention contemplates that miRNA-144 levels that are less than 50%, or less than 40%, or less than 30%, or less than 20% or less than 10%, or less than 5%, or 0% of such “maximum” control level (or range) may indicate that a subject will be responsive to and would benefit from IC or an IC-like intervention or miRNA-144 therapy at that time, particularly if the subject is experiencing a decrease in the level of miRNA-144 at the time. The contemplated IC or IC-like intervention may be an initial intervention or it may be a subsequent intervention (e.g., the subject may have already undergone an IC or IC-like intervention or miRNA-144 administration and the miRNA-144 levels are being measured to determine if and when to perform a subsequent intervention or administer a subsequent dose).

Other methods of the invention comprise performing IC or an IC-like intervention on a subject and measuring miRNA-144 level before and after IC or the IC-like intervention. Subjects responsive to IC or an IC-like intervention are identified by an increased level of miRNA-144 following IC or the IC-like intervention. Subjects not responsive to IC or an IC-like intervention are identified by steady state or decreased level of miRNA-144 following IC or an IC-like intervention.

The invention contemplates that separate and distinct control levels and ranges and time-courses can be established for each of IC, IC-like intervention, and miRNA-144 therapy. Thus a subject contemplated for treatment with IC may be compared to IC controls, a subject contemplated for treatment with IC-like intervention may be compared to IC-like controls, and a subject contemplated for treatment with miRNA-144 may be compared to miRNA-144 controls. The invention also contemplates that the control levels, ranges and time-courses may be established from combined datasets (i.e., data from IC and/or IC-like and/or miRNA-144 therapies, or any combination thereof).

The invention contemplates that miRNA levels may be measured in biological samples obtained from a subject. Suitable biological samples include but are not limited to whole blood, non-heparinized plasma, serum, urine, sputum, phlegm, saliva, tears, and other bodily fluids. In important embodiments, the biological sample is a whole blood sample or a serum sample derived therefrom.

miRNA are obtained from a biological sample using techniques used to harvest and/or isolate RNA generally. Harvest and isolation of total RNA from a sample is known in the art and reference can be made to standard RNA isolation protocols. (See, for example, Maniatis' Handbook of Molecular Biology.) The method does not require that miRNA be enriched from a standard RNA preparation. However, if desired, miRNA can be enriched using, for example, a YM-100 column.

miRNA-144 levels may be detected using any number of assays known in the art. These assays include miRNA arrays (including those that are commercially available from sources such as Agilent and Illumina), reverse transcriptase polymerase chain reaction (RT-PCR), quantitative real-time reverse transcriptase PCR (qPCR) using TaqMan microRNA assays (including those commercially available from sources such as Applied Biosystems, Foster City, Calif., USA), in situ hybridization, Northern hybridization, hybridization protection assay (HPA) (GenProbe), branched DNA (bDNA) assay (Chiron), rolling circle amplification (RCA), Invader assay (ThirdWave Technologies), and/or Oligo Ligation Assay (OLA), hybridization, and the like. A brief description of such methods are provided herein. Reference however can also be made to published US patent applications US2012/0165392 and US2013/0005658.

Some methods measure miRNA levels by amplifying all or part of miRNA nucleic acid sequences such as mature miRNAs, precursor miRNAs, and primary miRNAs. Suitable nucleic acid polymerization and amplification techniques include reverse transcription (RT), polymerase chain reaction (PCR), real-time PCR (quantitative PCR (q-PCR)), nucleic acid sequence-base amplification (NASBA), ligase chain reaction, multiplex ligatable probe amplification, invader technology (Third Wave), rolling circle amplification, in vitro transcription (IVT), strand displacement amplification, transcription-mediated amplification (TMA), RNA (Eberwine) amplification, and other methods that are known to persons skilled in the art. One or more amplification methods may be used, such as reverse transcription followed by real time PCR.

A typical PCR reaction includes multiple amplification steps, or cycles that selectively amplify target nucleic acid species. Since mature miRNAs are single stranded, a reverse transcription reaction (which produces a complementary cDNA sequence) is performed prior to PCR reactions. Reverse transcription reactions include the use of, e.g., a RNA-based DNA polymerase (reverse transcriptase) and a primer.

In PCR and q-PCR methods, for example, a set of primers is used for each target sequence. One primer (e.g., the forward primer) may comprise at least one sequence that anneals to a target miRNA and alternatively can comprise an additional 5′ non-complementary region. In another aspect, the other primer (e.g., the reverse primer) may anneal to the complement of a reverse transcribed miRNA. The reverse primer may be independent of the miRNA sequence, and multiple miRNAs may be amplified using the same reverse primer, for example if other miRNA are measured to test specificity of the assay. Alternatively, a reverse primer may be specific for miRNA-144.

In some embodiments, two or more miRNAs or nucleic acids are amplified in a single reaction volume or multiple reaction volumes. In certain aspects, one or more miRNA or nucleic may be used as a normalization control or a reference nucleic acid. Normalization may be performed in separate or the same reaction volumes as other amplification reactions. One aspect includes multiplex q-PCR, such as qRT-PCR, which enables simultaneous amplification and quantification of miRNA-144 and at least one reference nucleic acid in one reaction volume by using more than one pair of primers and/or more than one probe. The primer pairs comprise at least one amplification primer that uniquely binds each nucleic acid, and the probes are labeled such that they are distinguishable from one another, thus allowing simultaneous quantification of multiple miRNAs.

Real-time RT-PCR can be used to screen nucleic acids or RNA isolated from samples of interest and a related reference. A panel of targets including miRNA-144 is chosen for real-time RT-PCR measurement. The selection of the panel or targets can be based on the results of microarray analyses, such as mirVana™ miRNA Bioarray V1 (Ambion). A suitable normalization target may be 5S rRNA.

Certain aspects of the present invention concern the preparation and use of miRNA arrays or miRNA probe arrays, which are ordered macroarrays or microarrays of nucleic acid probes that are completely or nearly completely complementary or identical to one or more miRNAs such as miRNA-144 in mature or precursor form and are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters.

miRNA-144 as a Therapy

The invention contemplates the use of miRNA-144 as a prophylactic and/or therapeutic agent. As used herein, a prophylactic agent is an agent that is administered to a subject prior to the occurrence of an event, such as an ischemic event in order to reduce the likelihood that the event occurs, to prevent the occurrence of the event, to delay the onset of the event, and/or to reduce the severity associated with event. As used herein, a therapeutic agent is an agent that is administered to a subject during or after the occurrence of an event, such as an ischemic event, to reduce the severity of the event or its consequences.

The invention refers to administration of miRNA-144 generally. As used herein, administration of miRNA-144 refers generally to administration of miRNA-144 in its mature or precursor forms or in forms that are complementary in sequence to the mature and precursor forms. This may also be referred to herein as miRNA-144 therapy.

Such therapy includes nucleic acids that consist of or that comprise naturally occurring or non-naturally occurring miRNA-144 nucleotide sequence. Nucleic acids comprising the mature or precursor forms of miRNA-144 together with additional flanking nucleotides 5′ or 3′ to the miRNA-144 sequence may be used. The length of the nucleic acids may vary provided that they still achieve an IC-like effect when administered to a subject.

Certain aspects of the invention involve miRNAs having sequences are at least 80, 81, 82, 83, 84, 85, 86, 87, 88, 89, 90, 91, 92, 93, 94, 95, 96, 97, 98, 99% identical to the mature or precursor miRNA-144 sequence.

Naturally occurring miRNA-144 sequence is RNA in nature and comprises a phosphodiester backbone. Non-naturally occurring miRNA-144 sequence may comprise RNA elements (such as naturally occurring ribonucleotides) but it may also comprise non-naturally occurring elements (such as non-naturally occurring ribonucleotides or other nucleotide-like residues or backbone linkages other than phosphodiester linkages including but not limited to phosphorothioate linkages). Further examples of nucleotides, backbone linkages and other modifications are provided herein and may be incorporated into an miRNA-144 nucleic acid as contemplated by the invention. The Examples show the use of cholesterol modified residues and 2′-O-methyl modified oligonucleotides, for example. These nucleic acids may be administered directly or they may be formulated together with for example liposome or liposome-like coatings to prolong their half-life in vivo. Other formulations suitable for nucleic acid administration are known in the art (see for example published US patent applications US2012/0165392 and US2013/0005658) and may be used to deliver the miRNA-144 therapy contemplated by the invention. miRNA-144 therapy may also take the form of nucleic acids that are complementary to miRNA-144 mature and/or precursor sequences. This contemplates that miRNA-144 may be synthesized in vivo for short or long periods of time. One of ordinary skill is capable of designing such complementary sequences based on the knowledge of the miRNA-144 nucleotide sequence (mature and/or precursor) provided herein or otherwise known in the art. If miRNA-144 is to be synthesized in vivo, the subject may be administered a nucleic acid comprising its complementary sequence, optionally operably linked to regulatory nucleic acid sequences such as promoters and enhancers. The nucleic acids may be provided in vectors such as but not limited to viral vectors (e.g., adenovirus vectors).

Whether naturally occurring or non-naturally occurring, the nucleic acids may be isolated. This means that the nucleic acids are initially separated from different (in terms of sequence or structure) and unwanted nucleic acids and/or other moieties. In some instances, a population of isolated nucleic acids is at least about 90% homogenous, and may be at least about 95, 96, 97, 98, 99, or 100% homogenous with respect to sequence. In many embodiments of the invention, a nucleic acid is isolated because it has been synthesized in vitro separate from other nucleic acids.

In some embodiments, the nucleic acids administered to a subject do not contain miRNA-451 sequence whether in its mature or precursor form.

It will be appreciated based on the foregoing that miRNA-144 therapy may utilize single- and/or double-stranded nucleic acids.

miRNA-144 nucleic acids may be made using any technique known to one of ordinary skill in the art such as, for example, chemical synthesis, enzymatic production or biological production. It is specifically contemplated that miRNA probes of the invention are chemically synthesized. Non-limiting methods for synthesizing nucleic acids include in vitro chemical synthesis using phosphotriester, phosphite, or phosphoramidite chemistry and solid phase techniques such as described in EP 266,032, incorporated herein by reference, or using deoxynucleoside H-phosphonate intermediates as described by Froehler et al., 1986 and U.S. Pat. No. 5,705,629, each incorporated herein by reference. Various different mechanisms of oligonucleotide synthesis have been disclosed in for example, U.S. Pat. Nos. 4,659,774, 4,816,571, 5,141,813, 5,264,566, 4,959,463, 5,428,148, 5,554,744, 5,574,146, 5,602,244, each of which is incorporated herein by reference.

Reference can be made to published US patent applications US2012/0165392 and US2013/0005658 for methods and compositions that can be used to administer an miRNA-therapy, the specific teachings of which are incorporated herein by reference.

The invention relates, in part, to the administration of miRNA-144 to reduce or prevent ischemic/reperfusion injury in a subject. The invention is based, in part, on the unexpected and surprising finding that miRNA-144 administration is as effective as IC in protecting a tissue or organ from ischemic injury. IC including RIC has been shown to reduce ischemic and/or reperfusion injury associated with, inter alia, cardiac surgery, vascular surgery and myocardial infarction. The Examples demonstrate that the cardioprotective effects of IC are mediated, at least in part, through miRNA-144.

Thus, according to some aspects of the invention, miRNA-144 may be administered before, during and/or after an ischemic event. The subject may or may not have also undergone IC or an IC-like intervention before, during and/or after the ischemic event. The following describes the timing and frequency of miRNA-144 therapy in the context of a myocardial infarction. It is intended and should be understood that this description applies to other ischemic events and should be so construed.

When miRNA-144 is administered to a subject during, for example, a myocardial infarction, it may be administered prior to, or during the ischemia that is associated with a myocardial infarction (i.e., the ischemic phase or ischemic period), or during the reperfusion associated with a myocardial infarction (i.e., the reperfusion phase or reperfusion period), or during all phases to the same or to varying degrees.

When miRNA-144 is administered to a subject after, for example, a myocardial infarction, it may be administered within 30 minutes, within 1 hour, within 2 hours, within 3 hours, within 4 hours, within 5 hours, within 6 hours, within 8 hours, within 10 hours, within 12 hours, within 18 hours, or within 24 hours of the end of the ischemic phase of the myocardial infarction. In still other embodiments, miRNA-144 may be administered within 36 hours, 48 hours, or 60 hours of the myocardial infarction. The time between the myocardial infarction and the administration of miRNA-144 may be 1, 2, 3, 4, 5, or 6 days, or longer. miRNA-144 may be administered repeatedly to a subject over any time period including without limitation for up to 1 month, 2, 3, 4, 5, 6, 7, 8, 9, 10, 11, or 12 months, or longer following an MI. In some instances, it is administered over years including up to 2, 3, 4, 5, or more years. In still other instances, miRNA-144 is administered throughout the remaining lifespan of the subject.

It is to be understood that all of the foregoing teachings regarding timing relative to a myocardial infarction apply equally to any other ischemic event and that the invention embraces such other methods.

miRNA-144 may be administered on a daily basis, every other day (i.e., every two days), every three days, every four days, every five days, every six days, every week, or at longer intervals in time. In some instances, miRNA-144 may be administered at least once a day, at least once every two days, at least once every three days, at least once every four days, at least once every five days, at least once every six days, at least once every seven days following a myocardial infarction.

A subject includes but is not limited to humans and other non-human animals including, for example, companion animals such as dogs, cats, domesticated pigs, ferrets, hamsters, and the like; primates such as monkeys, and the like; agricultural animals such as cattle, pigs, horses, sheep, goats, birds (e.g., chickens, ducks, geese, and/or turkeys); prize-winning animals such as thoroughbreds, and the like. In important embodiments, the subject is a human subject.

### Ischemic/Reperfusion Injury

Ischemic and/or reperfusion injury, as used herein, refers to injury sustained in a subject's body due to ischemia and/or reperfusion associated with an ischemic event. The injury may be in any region of the body, including in any organ such as heart, kidney, liver, pancreas, lung, brain, intestine, spleen, and eyes. The subjects to be treated according to certain aspects of the invention are those that are likely to experience, have experienced, and/or are experiencing an ischemic event.

Ischemic and/or reperfusion injury, and thus reductions in ischemic and/or reperfusion injury, may be assessed anatomically and/or functionally, and the nature of such assessments will depend upon the region of the body or organ being treated or protected. As an example, an anatomical assessment of ischemic and/or reperfusion injury may be achieved through imaging and measuring observable tissue injury. As another example, a functional assessment of the ischemic and/or reperfusion injury may be achieved by measuring function of the affected tissue or organ. With respect to the heart, ischemic and/or reperfusion injury may be assessed for example by infarct size or it may be assessed through one or more hemodynamic parameters including for example ejection fraction. Imaging modalities such as computed tomography (CT), magnetic resonance (MR), arteriography, positron emission tomography (PET), and ultrasound including echocardiography can be used to assess a subject. Reducing ischemic and/or reperfusion injury, in some instances, provides long term benefits to a subject. As an example, reducing ischemic and/or reperfusion injury in the heart can ultimately lead to a reduction in the incidence of heart failure, or it can lead to a delay in the onset of heart failure, or it can reduce the severity of heart failure that develops. Accordingly, the invention provides methods for reducing ischemic and/or reperfusion injury that may be manifest in the short term or in the long term.

The invention contemplates protecting tissues and/or organs from ischemic and/or reperfusion injury or reducing the extent of such injury. It will be understood that the ischemic and/or reperfusion injury may exist in a variety of tissues and/or organs. Thus, while various aspects of the invention may be exemplified in the context of myocardial ischemia, the methods provided herein are broadly applicable to other types of tissue or organ ischemia as well.

The invention relates generally to the use of miRNA-144 as a companion diagnostic and/or a therapeutic agent in a variety of subjects including but not limited to those that are likely to experience, those that are experiencing, and/or those that have experienced an ischemic event in a tissue and/or organ of the body.

Ischemic events include but are not limited to cardiac ischemic events, cerebral ischemic events, renal ischemic events, pulmonary ischemic events, hepatic ischemic events, pancreatic ischemic events, ocular ischemic events, retinal ischemic events, intestinal ischemic events, and the like. Ischemic events also include acute ischemic conditions such as myocardial infarctions and strokes including transient ischemic stroke and hemorrhagic stroke, as well as chronic ischemic conditions. Ischemic events also include ischemia associated with or resulting from a surgery. The ischemia may occur during the surgery or it may occur after the surgery. Any surgery, regardless of location on the body, is associated with an increased risk of myocardial infarction and stroke post-surgery. This is particularly true in elderly subjects. This may be referred to as “consequential ischemia.” The surgery may be elective or emergency surgery, including but not limited to cardiovascular surgery including vascular surgery, cardiac surgery, stent placements such as intravascular stent placements, angioplasty such as balloon angioplasty, coronary artery bypass graft, heart valve surgery, heart transplantation, surgery for congenital heart disease, as well as lung surgery, liver surgery, kidney surgery, pancreas surgery, colon surgery, bowel surgery, including organ transplant such as but not limited to lung transplant, liver transplant, kidney transplant, and pancreas transplant.

The invention contemplates that short and/or long term benefits can be derived from administration of miRNA-144. For example, with respect to the heart, the methods of the invention provide short term benefits (e.g., the reduction of an infarct size) as well as long term benefits (e.g., the reduction in the likelihood and/or severity of heart failure, or delaying or preventing the onset of heart failure).

Generally, to treat, as used herein, encompasses to prevent, to delay, or to ameliorate, as appropriate, development or continuance or aggravation of a condition in a subject or to relieve, reduce or alleviate at least one symptom of a condition. For example, treatment can be diminishment of one or several symptoms of such a condition or complete eradication of the condition. Within the meaning of the present invention, the term “treat” also denotes to arrest, delay the onset (i.e., the period prior to clinical manifestation of a condition) and/or reduce the risk of developing or worsening a condition.

More specifically, to treat in the context of an ischemic event means to have a prophylactic or therapeutic benefit on a subject that is likely to experience, or that has experienced, or that is experiencing ischemic and/or reperfusion injury to a tissue and/or an organ. Typically this will involve a reduction in the injury which can be assessed in the short term and/or in the long term. An example of a short term assessment is infarct size resulting from an ischemic event (e.g., myocardial infarct size following a myocardial infarction). Another example of a short term assessment is hemodynamic function such as LVEDP and LVDP following an ischemic event such as a myocardial infarction.

The invention further contemplates that miRNA-144 may also reduce the likelihood, onset time, and/or severity of chronic injury resulting from the ischemic event and manifest in the long term. An example is congestive heart dysfunction/failure after a myocardial infarction. Such beneficial effects, in some instances, may be measured by comparing the subject to a population that has not been subjected to the methods of the invention. As an example, the subject and the “untreated” population can be compared in terms of incidence of heart dysfunction/failure, time of onset of heart dysfunction/failure, and severity of heart dysfunction/failure.

The invention contemplates use of miRNA therapy in a variety of subjects. The invention contemplates that any subject or any condition that is capable of deriving benefit from IC or IC-like intervention can be treated with miRNA-144 therapy as described herein. The following provides a description of certain conditions and subjects to be treated using miRNA-144. This list is not intended to be limiting and rather is intended as exemplary.

It is to be understood that miRNA-144 therapy may be used alone or in combination with IC, or IC-like invention, or other therapeutic agents, or any combination thereof.

### Myocardial Infarction

The invention contemplates the use of miRNA-144 as a companion diagnostic or as a therapeutic on subjects that have had one or more myocardial infarctions in the past (i.e., subjects with a history of myocardial infarction) and on subjects who have never knowingly had a myocardial infarction prior to being treated or screened according to the methods of the invention. These subjects may be treated according to the invention at the time of the myocardial infarction or shortly thereafter (e.g., within 6-12 hours of the myocardial infarction).

Those of ordinary skill in the art, including but not limited to medical practitioners and medical emergency personnel, will be familiar with the characteristics of an MI. Symptoms of MI, particularly in men, include sudden chest pain (often times radiating to the left arm or left side of neck), shortness of breath, nausea, vomiting, palpitations, sweating, and anxiety. Symptoms in women differ somewhat from those in men, and typically include shortness of breath, weakness, indigestion, and fatigue. Whether in the presence or absence of such symptoms, MI may be detected using, for example, electrocardiograms, blood marker tests (e.g., creatine-kinase, troponin T or I), and heart imaging such as chest X-rays. Guidelines for diagnosing an MI include the WHO criteria (i.e., history of ischemic type chest pain lasting for more than 20 minutes, changes in serial ECG tracings, and rise/fall of serum cardiac markers such as creatine kinase MB and troponin) in which the presence of two and three such criteria indicate probable and definite MI, respectively.

The invention contemplates the use of miRNA-144 during and/or after a myocardial infarction in order to reduce ischemic and/or reperfusion injury. A reduction in ischemic and/or reperfusion injury may be manifest as a reduction in the infarct size or volume following a myocardial infarction. The infarct size may be compared to infarct sizes from other comparable individuals or to infarct sizes from a population, including a population of comparable individuals that have not been treated according to the invention.

The subjects may also be monitored for their levels of serum markers such as but not limited to troponin, creatine kinase, serum potassium, serum sodium, and serum chloride.

Although not intending to limit the invention to any particular mechanism of action, it is contemplated that, when used in the context of a myocardial infarction, miRNA-144 may prevent or restrict the degree of left ventricular remodeling that would otherwise occur. miRNA-144 therapy, alone or in combination with IC (or an IC-like intervention) may attenuate inflammatory responses, reduce oxidative stress, and/or modulate hypertrophic and fibrotic signals associated with myocardial infarction.

It is also contemplated that therapeutic and long term benefits, such as a reduction in the incidence and/or severity of heart failure, may be had regardless of whether there is any observable reduction in infarct size.

Heart failure is generally defined as an impairment in the ability of the heart to pump blood through the body or to prevent blood from backing up into the lungs. Heart failure is often times referred to as congestive heart failure and is associated with systolic or diastolic heart dysfunction. It typically develops over time and may be triggered or exacerbated by another condition that causes heart tissue damage (e.g., an MI) or that causes the heart tissue to work more (or harder) than normal. Heart failure, as used herein, includes but is not limited to the complete cessation of pumping by the heart.

Accordingly, and as will be understood by those of ordinary skill in the art, heart failure indicates heart dysfunction and the invention contemplates reducing the risk, delaying the onset, preventing and/or treating heart dysfunction in the presence or absence of heart failure. The discussion of heart failure herein is therefore intended to capture heart dysfunction also, unless stated otherwise.

The invention provides, in some instances, methods for reducing the risk of heart dysfunction/failure in subjects who have had or are having an MI. The method is intended to reduce the development and/or severity of heart dysfunction/failure as a result of the MI. Development and severity of heart dysfunction/failure can be measured by monitoring and measuring symptoms or other characteristics associated with heart dysfunction/failure. These are discussed below. The methods may lead to the prevention of all or some such symptoms, the delayed onset of all or some such symptoms, and/or the reduction in the severity of all or some such symptoms. A reduction in the risk of heart dysfunction/failure may be determined by monitoring the symptoms or other characteristics associated with heart dysfunction/failure in the treated subject and comparing the number, onset, and severity of such symptoms or characteristics in that subject with historical population data for heart dysfunction/failure. For example, it is known that subjects that survive MI are more likely to develop heart dysfunction/failure than the average population. Some methods of the invention aim to reduce this likelihood or risk of heart dysfunction/failure development.

Symptoms of heart dysfunction/failure include shortness of breath (dyspnea), swelling in the feet and legs (edema) typically as a result of abnormal fluid retention, fluid in the lungs, persistent coughing or wheezing, low exercise tolerance, general fatigue even in the absence of exercise, increased heart rate (or palpitations), loss of appetite, memory loss (or confusion), and nausea. One and typically more than one of these symptoms will be manifest in subjects having heart dysfunction/failure. The methods of the invention aim to prevent the development, delay the onset, and/or reduce the severity of one or more of these symptoms.

Heart dysfunction/failure can be diagnosed based on presentation of one and typically more than one of the foregoing symptoms. Heart dysfunction/failure can also be diagnosed or a suspected diagnosis of heart dysfunction/failure can be confirmed with tests such as an electrocardiogram (ECG or EKG), an echocardiogram (“cardiac echo”), or cardiac catheterization. Echocardiograms, for example, are able to measure the volume or fraction of blood that is ejected from the left ventricle with each beat. This is referred to as the ejection fraction. In normal subjects, about 60% of the blood in the left ventricle is ejected. Subjects may present with mildly depressed ejection fractions (e.g., 40-45%), moderately depressed ejection fractions (e.g., 30-40%), or severely depressed ejection fractions (e.g., 10-25%). Thus, in some aspects of the invention, the methods aim to maintain the ejection fraction, particularly if the subject presents with normal or mildly depressed ejection fractions. In some aspects, the methods of the invention aim to delay the onset of a depressed ejection fraction, regardless of the initial ejection fraction presentation. Stress tests may also be used to diagnose heart dysfunction/failure, and they may be combined with one or more of the imaging tests discussed above. For example, a stress test may be combined with an echocardiogram in order to monitor and measure heart dysfunction/failure before, during and/or following exercise periods. Those of ordinary skill in the art, including medical practitioners and more particularly cardiologists, will be familiar these tests and their use in diagnosing heart dysfunction/failure.

### Cardiovascular Surgery

Some aspects of the invention comprise the use of miRNA-144 therapy to reduce ischemic and/or reperfusion injury resulting from cardiovascular surgery. The cardiovascular surgery may be performed on the heart and/or on the vasculature. Examples of cardiovascular surgery include but are not limited to heart transplantation, coronary artery by-pass surgery, cardiac valve surgery, surgery for congenital heart disease, carotid artery procedure, vascular grafting, vascular surgery including peripheral vascular surgery, and vascular replacement. Other minimally invasive procedures that are known to induce or likely to induce vessel damage are also considered ischemic events in the context of the invention, and these include stent placement and balloon angioplasty (or percutaneous transluminal coronary angioplasty (PTCA)). The vessel may be a blood vessel such as an artery or a vein.

The surgery or non-surgical procedure may be elective (and thus typically scheduled) or it may done on an emergency basis. The invention contemplates miRNA-144 therapy during and after, or after the surgery or procedure.

Stent placement or insertion may occur in any vessel of the body including many of the vessels discussed herein, and in any region of the body. Commonly, stent placement occurs intravascularly in an artery or in a vein. Stent placement may also occur in the bile duct, in the esophagus, and in the trachea. Stent placement may be used in any vessel to correct or ameliorate a narrowing of the vessel. The stents may be of any type, including “bare” stents (such as bare-metal stents, used as vascular stents) and drug-eluting stents. Drug-eluting stents, as used herein, refer to stents which are coated with or otherwise comprise one or more therapeutic agents. Bare stents, on the other hand, do not comprise such agents. Bare and drug-eluting stents are known in the art.

### Restenosis

Some aspects of the invention relate to the prevention or treatment of restenosis. Restenosis refers to renarrowing of a vessel or other narrowed biologic structure, and is a common complication following dilatation or stent placement (sometimes referred to as in stent restenosis). It can occur in anywhere from 10-50% of patients. miRNA-144 therapy may be used instead of, or in addition to, a surgical procedure to re-expand a narrowing.

Certain aspects of the invention provide for the use of miRNA-144 to reduce the occurrence and severity of restenosis. Restenosis may occur following a medical procedure (or intervention) aimed at opening or widening a blood vessel or biologic tube (including but not restricted to esophagus, biliary tree, bronchus, and the like). Such procedures include but are not limited to stent placements and balloon angioplasty, both of which can cause vessel damage.

miRNA-144 may be used as a companion diagnostic or as a therapeutic in a subject that has or that is likely to experience vessel damage that can lead to restenosis. In these subjects, miRNA-144 measurement and/or therapy may be occur before, during and/or after the occurrence of an event, such as a medical procedure, that is likely to induce vessel damage.

The subjects to be monitored and/or treated according to the invention include those that have undergone a medical intervention that induced or is likely to induce vessel damage. In some instances, these interventions do not themselves produce an ischemic event or environment in the subject.

Medical interventions that are known to induce or are likely to induce vessel damage may be any surgical or non-surgical procedure that results in damage to any vessel in the body. The vessel may be a blood vessel such as an artery or a vein. As used herein, the vessel may be a non-blood vessel (i.e., a vessel that carries a fluid other than, or in addition to, blood) such as the bile duct, the esophagus, the intestine (including large and small intestine), the trachea, the urethra, and the like.

An example of such an intervention is a stent placement (or insertion). Stent placement or insertion may occur in any vessel of the body including many of the vessels discussed herein, and in any region of the body. Commonly, stent placement occurs intravascularly in an artery or in a vein. Stent placement may also occur in the bile duct, in the esophagus, and in the trachea. Stent placement may be used in any vessel to correct or ameliorate a narrowing of the vessel.

The stents may be of any type, including “bare” stents (such as bare-metal stents, used as vascular stents) and drug-eluting stents. Drug-eluting stents, as used herein, refer to stents which are coated with or otherwise comprise one or more therapeutic agents. Bare stents, on the other hand, do not comprise such agents. Bare and drug-eluting stents are known in the art.

Another example of a medical intervention is angioplasty (or percutaneous transluminal coronary angioplasty (PTCA)). Restenosis has been reported to occur in 30-50% of subjects who have undergone simple balloon angioplasty.

Certain aspects of the invention intend to reduce the occurrence (or incidence) of restenosis in a subject, and/or to reduce the severity or degree of the restenosis, and/or to reduce or ameliorate the symptoms associated with restenosis.

A reduced occurrence of restenosis can be determined by comparing the treated subject to another subject, or more preferably a population of subjects, that has not received miRNA-144 therapy but is otherwise medically comparable to the treated subject. The average time of restenosis in this control group is compared to that of the treated subject, and a delayed onset of restenosis in the treated subject relative to the control is indicative of a reduced occurrence.

A reduction in the severity or degree of restenosis may be measured directly or indirectly. For example, the severity or degree of restenosis may be measured directly through, for example, measurement of a vessel diameter. Indirect measurements may include functional measurements. The nature of the functional measurement will depend upon the nature and normal function of the damaged vessel. An example of a functional measurement is flow rate and flow quality through the vessel. These measurements are preferably made when the restenosis is likely to occur, based on historical data from comparable but untreated subjects.

Analysis of symptoms relating to restenosis will also depend on the nature of the vessel(s) that may restenose. If restenosis may occur in the vasculature, then symptoms include any cardiovascular symptoms relating to blood flow impairment, including but not limited to cardiac and cerebral symptoms. These may include unusual fatigue, shortness of breath, and chest pressure.

Biological markers may also be measured as an indicator of restenosis. An example of a biological marker is troponin, which is elevated in the presence of restenosis.

Various tests are available to detect restenosis including imaging tests (e.g., CT, radionuclide imaging, angiography, Doppler ultrasound, MRA, etc.), and functional tests such as an exercise stress test.

### Traumatic Injury

The invention also provides methods for reducing the effects of trauma in subjects likely to experience trauma by administering miRNA-144 therapy prior to, during and/or following trauma. miRNA-144 therapy is intended to reduce the degree of injury in cells, one or more tissues and/or one or more organs that would be impacted by the trauma.

Thus, miRNA-144 therapy may be used to treat (including to ameliorate) the systemic effects associated with traumatic injury. Examples of traumatic injury that can be treated according to the invention include but are not limited to blunt trauma and hemorrhage (e.g., hemorrhagic shock).

The invention contemplates that miRNA-144 therapy will be administered to the subject by a first responder (i.e., the first qualified person to attend to the subject). The ability to achieve therapeutic benefit is invaluable in circumstances where other interventions, including intravenous fluid resuscitation, are not available or are delayed. These circumstances include without limitation battlefield conditions during military conflicts. Accordingly, the invention contemplates that miRNA-144 therapy can be used to reduce and/or prevent injury that is induced by trauma (e.g., hemorrhagic shock) in situations in which resuscitation therapy has not been performed, or was delayed, or is not yet complete. miRNA-144 therapy may be administered before the trauma, before resuscitation therapy, and/or after the resuscitation therapy, or any combination thereof. In like manner, the invention also contemplates administering miRNA-144 to a subject in preparation for a probable traumatic injury, including for example prior to military engagement or confrontation.

Subjects to be treated in this manner include those that are experiencing trauma and those that are likely to experience trauma. The ability to provide therapy to such subjects, particularly where there is no other therapy or intervention immediately available, is valuable. These methods can be used in other emergency situations in which no other therapy or intervention is immediately available such as can occur following catastrophic events such as earthquakes and other natural disasters, bombings, or in transport to a hospital or other critical care facility, and the like. Essentially, any subject that can experience traumatic injury can be treated according to the invention.

Trauma, as used herein, refers to critical or severe bodily injury, wound or shock. These forms of trauma typically require some form of resuscitation therapy. Resuscitation therapy typically involves replenishment of bodily fluids including but not limited to blood transfusion or other saline transfusion. Shock broadly refers to circulatory dysfunction. Shock may be hemorrhagic or hypovolemic shock (associated with inadequate blood volume) or it may be cardiogenic shock (associated with inadequate output of blood from the heart). Trauma associated with blood loss therefore typically also involves shock. Symptoms associated with shock include without limitation low blood pressure (i.e., hypotension), hypovolemia, hyperventilation, and cyanotic skin. In some instances, the trauma involves traumatic brain injury (e.g., the injury is to the head). In some instances, the trauma does not involve traumatic brain injury (e.g., the injury may be to the torso or one or more limbs).

miRNA-144 therapy may be administered to a subject that is hypovolemic and/or hypotensive. A subject that is hypovolemic may be a subject that has lost 5%, 10%, 15%, 20%, 25%, 30% or more of its whole blood volume. The cause of blood loss volume may be external bleeding, internal bleeding, or reduced blood volume resulting from excessive loss of other body fluids as may occur with diarrhea, vomiting and burns.

Trauma may result from direct injury such as penetrating injury (e.g., bullet wound). Trauma may also result from indirect injury such as, for example, a blast injury that occurs from exposure to a pressure wave following, for example, an explosion. Such latter types of trauma may occur in the absence of hypovolemia. In some instances, the invention contemplates the use of miRNA-144 therapy after traumatic injury not associated with hypovolemia. In these and other instances, miRNA-144 therapy may diminish systemic manifestations of the response to injury which includes neurologic injury and multi-organ dysfunction.

Since it is important to treat the subject as soon as possible, the invention contemplates that the methods provided herein may be performed in a hospital setting or in a non-hospital setting including in the environment in which the trauma occurred. miRNA-144 therapy may be administered before the trauma occurs, and/or after the trauma occurs, including before and/or after resuscitation therapy is performed. Repeated miRNA-144 therapy may be administered before, during and/or after trauma. In some embodiments, at least one miRNA-144 therapy is administered within about 48 hours, within about 24 hours, within about 12 hours, within about 6 hours, within about 4 hours, within about 2 hours, or within about 1 hour prior to trauma.

### Performance Enhancement

Other aspects of the invention are directed to the use of miRNA-144 as a companion diagnostic and/or active agent to enhance physical performance in subjects. These aspects of the invention are directed towards subjects who desire an improvement or enhancement of their level of physical activity or performance. Such subjects may not present with any diagnosed condition and may instead be regarded as healthy subjects.

In some instances, the invention is directed even more specifically to athletes, including competitive athletes. Such subjects are under a tremendous pressure to improve performance times and/or other judged end points without the use of prohibited performance enhancing drugs. The invention contemplates that IC (or IC-like intervention) would satisfy this need as it does not involve administration of any banned substance and instead simply takes advantage of inherent processes that operate in the body naturally. These subjects may be swimmers, short distance or long distance track runners, marathon runners, skiers, cyclists, and the like. miRNA-144 as a companion diagnostic can be used to identify subjects likely to be responsive to IC (or IC-like intervention). miRNA-144 as a companion diagnostic can also be used to stage or time the performance of one or more IC (or IC-like interventions) to achieve maximal effect and/or benefit.

These aspects of the invention are not limited solely to athletic subjects and instead can be applied to any subject that will perform a physical activity and in whom an improved performance is desired. The subjects may have average and possibly even below average athletic abilities yet would still be suited for the methods described herein. In some instances, the subjects are healthy. In some embodiments, the subjects may have poor heart function, heart failure, or other circulatory disturbances that might limit exercise performance. The subjects may or may not have angina including angina pectoris.

Such subjects will preferably be humans, although non-human subjects are also contemplated. Such non-human subjects include but again are not limited to any animal used in strenuous competition (e.g., racing) such as horses and dogs.

In one aspect, the invention provides a method for enhancing physical performance comprising administering miRNA-144 to a subject prior to a physical activity. In another aspect, the invention provides a method for enhancing physical performance comprising administering miRNA-144 to a healthy subject prior to a maximal physical activity. The methods of the invention can be used as a long-term training regimen.

In some embodiments, miRNA is administered to a subject prior to and typically not during the physical activity. It may be administered within 48 hours, within 24 hours, within 12 hours, within 6 hours, within 4 hours, within 2 hours, within 1 hour, within 30 minutes, within 20 minutes, within 10 minutes, or within 5 minutes prior to the physical activity, or just immediately prior to the physical activity. It may be administered one or more times, in one day, or per day (daily), or on prescribed days over the course of days, weeks, or months.

In some aspects of the invention, the method is intended to improve the performance of a maximal physical activity. As used herein, the term maximal physical activity means an activity in which the subject exerts itself maximally. Exertion levels may be measured in a number of ways known in the art including but not limited to heart rate range, the “talk test”, and the Borg rating of perceived exertion (RPE). The degree of activity that yields maximal exertion may vary between certain subjects based on age and physical condition. Nevertheless, methods exist in the art to determine for each subject the level of activity that corresponds to moderate, vigorous or maximal exertion.

The following is a method for determining the level of activity being performed for a given individual using heart rate. Generally, the person's age is subtracted from the hypothetical maximum heart rate of 220. The resulting number is multiplied by a percentage based upon the level of activity being performed. Moderate intensity activity corresponds to about 50-70% of the “age-adjusted” maximum heart rate. Vigorous intensity activity corresponds to 70-85% of the “age-adjusted” maximum heart rate. Maximal activity corresponds to anything higher than 85% of the age-adjusted maximum heart rate.

If the Borg RPE is used, a score of 19 or 20 corresponds to maximal exertion, a score in the range of 15-18 corresponds to vigorous exertion, and a score in the range of 12-14 corresponds to moderate exertion.

In still other embodiments, particularly those which involve subjects with cardiovascular disease, exercise may be limited. In these and other similar situations, an exercise intensity level of NYHA (New York Heart Association) grade 2-4 is contemplated.

Examples of moderate intensity activity include but are not limited to walking briskly (3 miles per hour or faster), water aerobics, bicycling slower than 10 miles per hour, ballroom dancing, tennis (doubles), and general gardening.

Examples of vigorous intensity activity include but are not limited to race walking, jogging or running (e.g., marathon running or racing), swimming laps, tennis (singles), aerobic dancing, bicycling 10 mile per hour or faster, biathlons, triathlons, or other single or multiple activity competitions (e.g., Iron Man competitions), diving such as deep sea diving, free diving and base diving, jumping rope, heavy gardening (e.g., continuous digging or hoeing), hiking uphill or with a heavy backpack, and the like.

The activity to be benefited according to the invention may be short (e.g., 60 minutes or less, including 5, 10, 20, 30, 40, 50 or more minutes) or it may be long (e.g., more than one hour, including 2, 3, 4, 5, 6 or more hours) in duration.

Physical activity that can also benefit from the methods of the invention includes the activity associated with a rescue operation such as a coast guard rescue operation (e.g., a rescue at sea), activity associated with first-responder activity (e.g., rescuing persons from a burning building), activity associated with hand-to-hand combat military missions, and the like.

Maximal intensity activity could typically be any of the vigorous intensity activities recited herein provided they are performed at the individual subject's maximal ability (i.e., an “all-out” attempt).

It is to be understood that the invention provides methods for improving performance that occurs for any of the foregoing activities since whether a particular activity will require moderate, vigorous or maximum exertion will depend on the individual and their physical ability and condition.

It is also to be understood that the invention contemplates using miRNA-144 to enhance submaximal activities also. The invention contemplates that subjects less physically fit than competitive athletes will also benefit from miRNA-144 administration, for example, when performing submaximal activity.

The methods for measuring performance enhancement will vary based on the particular activity being performed. For example, if the activity is swimming, then the enhancement may be measured by the time to swim a certain distance (e.g., 50 meters, 100 meters, or more). If the activity is running, then the enhancement may be measured by the time to run a certain distance (e.g., 50 meters, 100 meters, 200 meters, 1 mile, a marathon, etc.). Similarly, if the activity is cycling, speed skating, and the like, then the enhancement may be measured by the time taken to traverse a certain distance. It will be understood that in these examples, the enhancement will be manifested as a decrease in the time taken to perform the activity in question. Other suitable endpoints and readouts will be apparent to those of ordinary skill in the art.

The degree of performance enhancement that can be achieved using the methods provided herein may vary between individuals. The degree of performance enhancement will typically be measured using the difference between the endpoints or readouts achieved following miRNA-144 administration and a sham control. The quotient of that difference and the sham control readout is representative of the improvement achieved. As an example, a 1% enhancement is a decrease of a second for an activity that would take on average 100 seconds to perform in the absence of miRNA-144 administration.

In some instances, the degree of enhancement may be on the order of 0.1%-1%, including 0.5%-1% yet still be statistically significant and more importantly competitive or physiologically significant. In still other instances, the degree of enhancement may be up to 1.5%, up to 2%, up to 2.5%, up to 3%, up to 3.5%, up to 4%, up to 4.5%, up to 5%, up to 10%, up to 20%, up to 30%, up to 40%, up to 50%, or more.

Accordingly, various aspects of the invention provide methods to improve resistance to exercise-induced fatigue in healthy individuals during sports and activities, and in patients limited by cardiac, circulatory or other medical disorders (e.g., patients with heart failure, peripheral vascular disease, lung disease) that may limit blood flow or muscle power.

### Cancers

The invention contemplates use of miRNA-144 based methods in subjects having or at risk of developing cancer. Such cancers include but are not limited to basal cell carcinoma, biliary tract cancer; bladder cancer; bone cancer; brain cancer; breast cancer; cervical cancer; choriocarcinoma; CNS cancer; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; acute myeloid leukemia; acute lymphoid leukemia, chronic myeloid leukemia, chronic lymphoid leukemia, leukemia, liver cancer; small cell lung cancer; non-small cell lung cancer; lymphoma, Hodgkin's lymphoma; Non-Hodgkin's lymphoma; melanoma; myeloma; neuroblastoma; oral cavity cancer; ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; and cancer of the urinary system.

In another embodiment, the cancer is selected from the group consisting of bladder cancer, breast cancer, colon cancer, endometrial cancer, head and neck cancer, leukemia, lung cancer, lymphoma, melanoma, ovarian cancer, prostate cancer and rectal cancer.

In another embodiment, the cancer is a refractory cancer. Examples of refractory cancers include but are not limited to leukemias, melanomas, renal cell carcinomas, colon cancer, liver (hepatic) cancers, pancreatic cancer, Non-Hodgkin's lymphoma, and lung cancer. In still other embodiments, the cancer is an immunogenic cancer.

In still another embodiment, the cancer is a metastasis.

### Neurodegenerative Diseases

The invention contemplates use of miRNA-144 based methods in subjects having or at risk of developing a neurodegenerative disease. Neurodegenerative diseases include but are not limited to Alzheimer's disease, Huntington's disease, multiple sclerosis, and Parkinson's disease.

### Infectious Diseases

The invention contemplates use of miRNA-144 based methods in subjects having an infectious disease. The infectious disease may be selected from the group consisting of a bacterial infection, a mycobacterial infection, a viral infection, a fungal infection and a parasitic infection, but it is not so limited.

In one embodiment, the bacterial infection is selected from the group consisting of an E. coli infection, a Staphylococcal infection, a Streptococcal infection, a Pseudomonas infection, Clostridium difficile infection, Legionella infection, Pneumococcus infection, Haemophilus infection, Klebsiella infection, Enterobacter infection, Citrobacter infection, Neisseria infection, Shigella infection, Salmonella infection, Listeria infection, Pasteurella infection, Streptobacillus infection, Spirillum infection, Treponema infection, Actinomyces infection, Borrelia infection, Corynebacterium infection, Nocardia infection, Gardnerella infection, Campylobacter infection, Spirochaeta infection, Proteus infection, Bacteroides infection, H. pylori infection, and anthrax infection.

The mycobacterial infection may be tuberculosis or leprosy respectively caused by the M. tuberculosis and M. leprae species, but is not so limited.

In one embodiment, the viral infection is selected from the group consisting of an HIV infection, a Herpes simplex virus 1 infection, a Herpes simplex virus 2 infection, cytomegalovirus infection, hepatitis A virus infection, hepatitis B virus infection, hepatitis C virus infection, human papilloma virus infection, Epstein Barr virus infection, rotavirus infection, adenovirus infection, influenza A virus infection, respiratory syncytial virus infection, varicella-zoster virus infections, small pox infection, monkey pox infection and SARS infection.

In yet another embodiment, the fungal infection selected from the group consisting of candidiasis, ringworm, histoplasmosis, blastomycosis, paracoccidioidomycosis, crytococcosis, aspergillosis, chromomycosis, mycetoma infections, pseudallescheriasis, and tinea versicolor infection.

In another embodiment, the parasite infection is selected from the group consisting of amebiasis, Trypanosoma cruzi infection, Fascioliasis, Leishmaniasis, Plasmodium infections, Onchocerciasis, Paragonimiasis, Trypanosoma brucei infection, Pneumocystis infection, Trichomonas vaginalis infection, Taenia infection, Hymenolepsis infection, Echinococcus infections, Schistosomiasis, neurocysticercosis, Necator americanus infection, and Trichuris trichiura infection.

In another embodiment, the infectious disease is an infection of a Mycobacteria sps (e.g. M. tuberculosis, M. avium, M. intracellulare, M. kansaii, M. gordonae), Shigella flexneri, salmonella enterica, listeria monocytogenes, and francisella tularensis.

### Gastrointestinal Diseases

The invention contemplates use of miRNA-144 based methods in subjects having or at risk of developing a gastrointestinal condition. Gastrointestinal conditions include but are not limited to such as Crohn's disease and ulcerative colitis.

### Cardiovascular Diseases

The invention contemplates use of miRNA-144 based methods in subjects having or at risk of developing a cardiovascular disease, including atherosclerosis, cardiomyopathies, cardiac hypertrophy, ischemic heart disease, heart failure, and ischemia reperfusion injury.

### Other Conditions

The invention contemplates use of miRNA-144 based methods in subjects having or at risk of developing a genetic x-linked lysosome associated membrane protein disease such as Danon's disease, mitochondrial myopathies, and chronic myocarditis.

The invention contemplates use of miRNA-144 based methods in subjects having or at risk of developing metabolic diseases or conditions such as but not limited to insulin sensitivity and diabetes, obesity, metabolic syndrome, glucose intolerance, hyperlipidemia, and hypercholesterolemia.

The invention contemplates use of miRNA-144 based methods in subjects having or at risk of developing pulmonary diseases such as chronic obstructive pulmonary disease, cystic fibrosis, emphysema, asthma, pulmonary hypertension, and idiopathic pulmonary fibrosis.

### Additional Therapies

In some aspects of the invention, miRNA-144 therapy may be used in combination with other therapies or procedures. Some additional therapies involve administration of a second active agent to a subject. Some additional procedures involve performing IC or an IC-like intervention on a subject.

### Ischemic Conditioning (IC)

Some methods of the invention are intended to determine whether an IC intervention is likely to be efficacious in a subject and/or the degree of efficacy achieved in a subject using IC. IC, as used herein, refers to a deliberately induced ischemic event or period followed by a reperfusion event or period. IC may be performed as a single cycle (i.e., one ischemic event followed by one reperfusion event) or as multiple cycles. Multiple cycles include but are not limited to two, three, four, five or more cycles. IC may be performed locally or remotely. Local IC involves blood flow occlusion and reperfusion in a tissue or organ or region of the body to be protected from an existing or a future anticipated ischemia/reperfusion injury. An example is local IC of the heart prior to cardiac surgery. Remote IC (RIC) involves blood flow occlusion and reperfusion in a tissue or organ or region of the body that is remote to the region of the body to be protected. Remote IC is typically performed in a lower or upper. IC is preferably non-invasive.

The blood flow restriction (or occlusion) typically takes the form of an applied pressure to the limb or tissue that is sufficient to occlude blood through the limb or through and/or to the tissue. In some instances, the occlusive blood pressure is above systolic pressure (i.e., supra-systolic pressure). It may be about 5, about 10, about 15, about 20, or more mmHg above (or greater than) systolic pressure. In some instances, the occlusive blood pressure may be at or below systolic pressure. Since systolic pressure will differ between subjects, the absolute pressure needed to induce ischemia will vary between subjects. In other embodiments the pressure may be preset at, for example, 200 mmHg. The blood flow restriction may be accomplished using any method or device provided it is capable of inducing transient ischemia and reperfusion, whether manually or automatically. Such devices include without limitation a manually inflatable cuff, a tourniquet system, or an automated device as described below. The devices comprise cuffs of standard width or cuffs of greater than standard width.

The induced ischemic event is transient. That is, it may have a duration of about 1, about 2, about 3, about 4, about 5, or more minutes. Similarly, the reperfusion event may have a duration of about 1, about 2, about 3, about 4, about 5, or more minutes.

If performed using a limb, one or both upper limbs or one or both lower limbs may be used although in some instances one or both upper limbs is preferred. In some instances, IC is performed on two different sites on the body, in an overlapping or simultaneous manner.

Devices for performing RIC are also known in the art, and include those described in U.S. Pat. No. 7,717,855 and US patent application publication 2012/0265240 A1, both of which are incorporated herein by reference in their entirety. Briefly, this system comprises a cuff configured to retract about a limb of a subject, an actuator connected to the cuff that when actuated causes the cuff to contract about the limb of the subject to reduce blood flow therethrough, and a controller that controls the actuator according to a treatment protocol. The treatment protocol typically includes a plurality of treatment cycles, each of which may comprise a cuff actuation period during which the actuator contracts the cuff about the limb of the subject to a pressure that occludes blood flow through the limb, an ischemic duration period during which the actuator maintains the cuff contracted about the limb at a set pressure point to occlude blood flow through the limb, a cuff release period during which the actuator releases the cuff to allow blood flow through the limb, and a reperfusion duration period during which the cuff is maintained about the limb in a relaxed state to allow blood flow through the limb.

### IC-Like Interventions

The methods provided herein also can be used to identify a subject that is likely (or unlikely) to respond to an IC-like intervention. IC-like interventions include but are not limited to non-invasive electrical nerve stimulation such as transcutaneous electrical nerve stimulation, direct nerve stimulation such as femoral nerve stimulation, electro-acupuncture, nociceptive c-fiber stimulation for example via topical capsaicin, intra-arterial adenosine, and vigorous exercise.

As used herein, non-invasive electrical nerve stimulation may be a single cycle of nerve stimulation followed by a rest period during which no current is applied to the subject, or it may be repeated cycles of nerve stimulation followed by a rest period. The repeated cycles may comprise 2, 3, 4, 5 or more cycles of nerve stimulation followed by a rest period. For clarity, two cycles of non-invasive electrical nerve stimulation would consist of a nerve stimulation period, a rest period, a nerve stimulation period, and a rest period. The invention contemplates that, in some embodiments, a single nerve stimulation period may be sufficient to achieve the desired therapeutic, prophylactic or performance endpoints.

The nerve stimulation period and the rest period may each range from 30 seconds to several minutes or hours. Either or both periods may be up to or about 30 seconds, or 1, 2, 3, 4, 5, 6, 7, 8, 9, 10, 15, 20, 25, 30, 35, 40, 45, 50, 55 or 60 minutes in duration, or longer. The two periods may or may not be of the same duration. An exemplary non-invasive electric nerve stimulation comprises 4 or 5 cycles of 5 minutes of nerve stimulation followed by 5 minutes of rest. Another exemplary non-invasive electrical nerve stimulation comprises 4 or 5 cycles of 4 minutes of nerve stimulation followed by 4 minutes of rest. The non-invasive electrical nerve stimulation device may be operated under any number of pulse amplitude (or intensity), pulse width, and pulse frequency settings. As an example, the pulse amplitude may range from 1 to 200 mA, including typically from 1 to 100 mA, from 1 to 90 mA, from 1-80 mA, from 1-70 mA, from 1-60 mA, from 1-50 mA, from 1-40 mA, from 1-30 mA, from 1-20 mA, from 1-15 mA, from 1-10 mA, from 1-9 mA, from 1-8 mA, from 1-7 mA, from 1-6 mA, from 1-5 mA, from 1-4 mA, from 1-3 mA, or from 1-2 mA. The pulse frequency may range from 1 to 300 Hz, including typically from 1 to 150 Hz, from 1-140 Hz, from 1-130 Hz, from 1-120 Hz, from 1-110 Hz, from 1-100 Hz, from 1-90 Hz, from 1-80 Hz, from 1-70 Hz, from 1-60 Hz, from 1-50 Hz, from 1-40 Hz, from 1-30 Hz, from 1-20 Hz, from 1-10 Hz, from 1-9 Hz, from 1-8 Hz, from 1-7 Hz, from 1-6 Hz, from 1-5 Hz, from 1-4 Hz, from 1-3 Hz, or from 1-2 Hz. The pulse width may range up to 1 to 1600 microseconds, including typically from 1 to 800 microseconds, from 1-700 milliseconds, from 1-600 milliseconds, from 1-500 milliseconds, from 1-400 milliseconds, from 1-300 milliseconds, from 1-200 milliseconds, from 1-100 milliseconds, and from 1-50 milliseconds. The device may also operate at a voltage typically up to 80 V, including typically up to 40 V, up to 30 V, up to 20 V, up to 10 V, and up to 5 V. The Examples show exemplary settings in which the pulse amplitude is 2-3 mA, the pulse frequency is 3.1 Hz, and the pulse width is 500 microseconds.

Non-invasive electrical nerve stimulation may be performed at any site on the body that is amenable to the non-invasive procedure. It may be performed on any outer surface of the body, including but not limited to arms, legs, feet, hands, torso, chest, back, and the like. In some embodiments, it is performed on the abdomen. In some embodiments it is performed on regions of the body other than the abdomen. It may be performed at a remote site (i.e., a site that is distal to the area of the body experiencing or likely to experience the ischemic and/or reperfusion injury). In other words, the placement of the electrodes may be distal to the region of the body being treated. As an example, the electrodes may be placed on the legs in order to reduce injury in the heart. Typically at least two electrodes are placed within proximity of each other in order to allow current to flow therebetween. Additional paired electrodes may be used at the same or different surface region of the body at the same or different time.

Repeated non-invasive electrical nerve stimulations may be performed at a single, identical site or at multiple, different sites on the body. As an example, a first stimulation may be performed on the right upper arm, followed by a second stimulation performed on the left upper arm. In some embodiments, the non-invasive electrical nerve stimulation is not performed on the chest. Repeated non-invasive electrical nerve stimulations may alternate between two sites or they may cycle through more than two sites. In some instances, non-invasive electrical nerve stimulation may be performed on a subject at two different sites at overlapping times including simultaneously. The use of more than one location may be determined a priori or it may be random. When multiple locations are used simultaneously, two or more devices are typically used.

### Secondary Prophylactic or Therapeutic Therapies

miRNA-144 therapy may be used, in some instances, to reduce the risk or severity of heart damage and/or heart dysfunction/failure. In these instances, miRNA-144 therapy may be used with a secondary therapy such as but not limited to anti-platelet drug therapy including fibrinolytic agents, anti-coagulation agents, and platelet function inhibitors, beta blocker therapy, ACE inhibitor therapy, statin therapy, aldosterone antagonist therapy (e.g., eplerenone), and omega-3-fatty acids therapy. Depending upon the embodiment, one or more of these agents may be administered before, at the time of, or after MI, whether or not overlapping with the miRNA-144 therapy. These and other suitable therapies are discussed in greater detail below.

Fibrinolytic agents are agents that lyse a thrombus (e.g., a blood clot), usually through the dissolution of fibrin by enzymatic action. Examples include but are not limited to ancrod, anistreplase, bisobrin lactate, brinolase, Hageman factor (i.e. factor XII) fragments, molsidomine, plasminogen activators such as streptokinase, tissue plasminogen activators (TPA) and urokinase, and plasmin and plasminogen.

Anti-coagulant agents are agents that inhibit the coagulation pathway by impacting negatively upon the production, deposition, cleavage and/or activation of factors essential in the formation of a blood clot. Anti-coagulant agents include but are not limited to vitamin K antagonists such as coumarin and coumarin derivatives (e.g., warfarin sodium); glycosoaminoglycans such as heparins both in unfractionated form and in low molecular weight form; ardeparin sodium, bivalirudin, bromindione, coumarin dalteparin sodium, desirudin, dicumarol, lyapolate sodium, nafamostat mesylate, phenprocoumon, sulfatide, tinzaparin sodium, inhibitors of factor Xa, factor TFPI, factor VIIa, factor IXc, factor Va, factor VIIIa as well as inhibitors of other coagulation factors.

Inhibitors of platelet function are agents that impair the ability of mature platelets to perform their normal physiological roles (i.e., their normal function). Examples include but are not limited to acadesine, anagrelide, anipamil, argatroban, aspirin, clopidogrel, cyclooxygenase inhibitors such as nonsteroidal anti-inflammatory drugs and the synthetic compound FR-122047, danaparoid sodium, dazoxiben hydrochloride, diadenosine 5′,5′″-P1,P4-tetraphosphate (Ap4A) analogs, difibrotide, dilazep dihydrochloride, 1,2- and 1,3-glyceryl dinitrate, dipyridamole, dopamine and 3-methoxytyramine, efegatran sulfate, enoxaparin sodium, glucagon, glycoprotein IIb/IIIa antagonists such as Ro-43-8857 and L-700,462, ifetroban, ifetroban sodium, iloprost, isocarbacyclin methyl ester, isosorbide-5-mononitrate, itazigrel, ketanserin and BM-13.177, lamifiban, lifarizine, molsidomine, nifedipine, oxagrelate, PGE, platelet activating factor antagonists such as lexipafant, prostacyclin (PGI₂), pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612 and ZK-36374,2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as picotamide and sulotroban, ticlopidine, tirofiban, trapidil and ticlopidine, trifenagrel, trilinolein, 3-substituted 5,6-bis(4-methoxyphenyl)-1,2,4-triazines, and antibodies to glycoprotein IIb/IIIa as well as those disclosed in U.S. Pat. No. 5,440,020, and anti-serotonin drugs, Clopridogrel; Sulfinpyrazone; Aspirin; Dipyridamole; Clofibrate; Pyridinol Carbamate; PGE; Glucagon; Antiserotonin drugs; Caffeine; Theophyllin Pentoxifyllin; Ticlopidine.

In some instances, the therapies or procedures are used to reduce inflammation associated with certain conditions such as restenosis. Anti-inflammatory agents include without limitation Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Morniflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Salycilates; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Glucocorticoids; Zomepirac Sodium. One preferred anti-inflammatory agent is aspirin.

Lipid reducing agents include gemfibrozil, cholystyramine, colestipol, nicotinic acid, probucol lovastatin, and statins such as fluvastatin, simvastatin, atorvastatin, pravastatin, and cirivastatin.

Direct thrombin inhibitors include hirudin, hirugen, hirulog, agatroban, PPACK, thrombin aptamers.

Glycoprotein IIb/IIIa receptor inhibitors are both antibodies and non-antibodies, and include but are not limited to ReoPro (abcixamab), lamifiban, tirofiban.

Calcium channel blockers are a chemically diverse class of compounds having important therapeutic value in the control of a variety of diseases including several cardiovascular disorders, such as hypertension, angina, and cardiac arrhythmias (Fleckenstein, Cir. Res. v. 52, (suppl. 1), p. 13-16 (1983); Fleckenstein, Experimental Facts and Therapeutic Prospects, John Wiley, New York (1983); McCall, D., Curr Pract Cardiol, v. 10, p. 1-11 (1985)). Calcium channel blockers are a heterogeneous group of drugs that prevent or slow the entry of calcium into cells by regulating cellular calcium channels. (Remington, The Science and Practice of Pharmacy, Nineteenth Edition, Mack Publishing Company, Eaton, Pa., p. 963 (1995)). Most of the currently available calcium channel blockers, and useful according to the present invention, belong to one of three major chemical groups of drugs, the dihydropyridines, such as nifedipine, the phenyl alkyl amines, such as verapamil, and the benzothiazepines, such as diltiazem. Other calcium channel blockers useful according to the invention, include, but are not limited to, amrinone, amlodipine, bencyclane, felodipine, fendiline, flunarizine, isradipine, nicardipine, nimodipine, perhexilene, gallopamil, tiapamil and tiapamil analogues (such as 1993RO-11-2933), phenytoin, barbiturates, and the peptides dynorphin, omega-conotoxin, and omega-agatoxin, and the like and/or pharmaceutically acceptable salts thereof.

Beta-adrenergic receptor blocking agents (also known as beta blockers) are a class of drugs that antagonize the cardiovascular effects of catecholamines in angina pectoris, hypertension, and cardiac arrhythmias. Beta-adrenergic receptor blockers include, but are not limited to, atenolol, acebutolol, alprenolol, befunolol, betaxolol, bunitrolol, carteolol, celiprolol, hedroxalol, indenolol, labetalol, levobunolol, mepindolol, methypranol, metindol, metoprolol, metrizoranolol, oxprenolol, pindolol, propranolol, practolol, practolol, sotalolnadolol, tiprenolol, tomalolol, timolol, bupranolol, penbutolol, trimepranol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4-(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide. The above-identified compounds can be used as isomeric mixtures, or in their respective levorotating or dextrorotating form.

A number of selective “COX-2 inhibitors” are known in the art. These include, but are not limited to, COX-2 inhibitors described in U.S. Pat. No. 5,474,995 “Phenyl heterocycles as cox-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-benzylindol-3-yl propanoic acid derivatives as cyclooxygenase inhibitors”; U.S. Pat. No. 5,604,260 “5-methanesulfonamido-1-indanones as an inhibitor of cyclooxygenase-2”; U.S. Pat. No. 5,639,780 N-benzyl indol-3-yl butanoic acid derivatives as cyclooxygenase inhibitors“; U.S. Pat. No. 5,677,318 Diphenyl-1,2-3-thiadiazoles as anti-inflammatory agents”; U.S. Pat. No. 5,691,374 “Diaryl-5-oxygenated-2-(5H)-furanones as COX-2 inhibitors”; U.S. Pat. No. 5,698,584 “3,4-diaryl-2-hydroxy-2,5-dihydrofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif.). Additional COX-2 inhibitors are also described in U.S. Pat. No. 5,643,933, assigned to G. D. Searle & Co. (Skokie, Ill.), entitled: “Substituted sulfonylphenylheterocycles as cyclooxygenase-2 and 5-lipoxygenase inhibitors.”

A number of the above-identified COX-2 inhibitors are prodrugs of selective COX-2 inhibitors, and exert their action by conversion in vivo to the active and selective COX-2 inhibitors. The active and selective COX-2 inhibitors formed from the above-identified COX-2 inhibitor prodrugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present invention.

An angiotensin system inhibitor is an agent that interferes with the function, synthesis or catabolism of angiotensin II. These agents include, but are not limited to, angiotensin-converting enzyme (ACE) inhibitors, angiotensin II antagonists, angiotensin II receptor antagonists, agents that activate the catabolism of angiotensin II, and agents that prevent the synthesis of angiotensin I from which angiotensin II is ultimately derived. The renin-angiotensin system is involved in the regulation of hemodynamics and water and electrolyte balance. Factors that lower blood volume, renal perfusion pressure, or the concentration of Na⁺ in plasma tend to activate the system, while factors that increase these parameters tend to suppress its function.

Angiotensin II antagonists are compounds which interfere with the activity of angiotensin II by binding to angiotensin II receptors and interfering with its activity. Angiotensin II antagonists are well known and include peptide compounds and non-peptide compounds. Most angiotensin II antagonists are slightly modified congeners in which agonist activity is attenuated by replacement of phenylalanine in position 8 with some other amino acid; stability can be enhanced by other replacements that slow degeneration in vivo. Examples of angiotensin II antagonists include but are not limited to peptidic compounds (e.g., saralasin, [(San¹)(Val⁵)(Ala⁸)] angiotensin-(1-8) octapeptide and related analogs); N-substituted imidazole-2-one (U.S. Pat. No. 5,087,634); imidazole acetate derivatives including 2-N-butyl-4-chloro-1-(2-chlorobenzile) imidazole-5-acetic acid (see Long et al., J. Pharmacol. Exp. Ther. 247(1), 1-7 (1988)); 4,5,6,7-tetrahydro-1H-imidazo[4, 5-c]pyridine-6-carboxylic acid and analog derivatives (U.S. Pat. No. 4,816,463); N2-tetrazole beta-glucuronide analogs (U.S. Pat. No. 5,085,992); substituted pyrroles, pyrazoles, and tryazoles (U.S. Pat. No. 5,081,127); phenol and heterocyclic derivatives such as 1,3-imidazoles (U.S. Pat. No. 5,073,566); imidazo-fused 7-member ring heterocycles (U.S. Pat. No. 5,064,825); peptides (e.g., U.S. Pat. No. 4,772,684); antibodies to angiotensin II (e.g., U.S. Pat. No. 4,302,386); and aralkyl imidazole compounds such as biphenyl-methyl substituted imidazoles (e.g., EP Number 253,310, Jan. 20, 1988); ES8891 (N-morpholinoacetyl-(-1-naphthyl)-L-alanyl-(4, thiazolyl)-L-alanyl (35, 45)-4-amino-3-hydroxy-5-cyclo-hexapentanoyl-N-hexylamide, Sankyo Company, Ltd., Tokyo, Japan); SKF108566 (E-alpha-2-[2-butyl-1-(carboxy phenyl)methyl]1H-imidazole-5-yl[methylane]-2-thiophenepropanoic acid, Smith Kline Beecham Pharmaceuticals, PA); Losartan (DUP753/MK954, DuPont Merck Pharmaceutical Company); Remikirin (RO42-5892, F. Hoffman LaRoche AG); A₂ agonists (Marion Merrill Dow) and certain non-peptide heterocycles (G.D. Searle and Company).

ACE inhibitors include amino acids and derivatives thereof, peptides, including di- and tri-peptides and antibodies to ACE which intervene in the renin-angiotensin system by inhibiting the activity of ACE thereby reducing or eliminating the formation of pressor substance angiotensin II. ACE inhibitors have been used medically to treat hypertension, congestive heart dysfunction/failure, myocardial infarction and renal disease. Classes of compounds known to be useful as ACE inhibitors include acylmercapto and mercaptoalkanoyl prolines such as captopril (U.S. Pat. No. 4,105,776) and zofenopril (U.S. Pat. No. 4,316,906), carboxyalkyl dipeptides such as enalapril (U.S. Pat. No. 4,374,829), lisinopril (U.S. Pat. No. 4,374,829), quinapril (U.S. Pat. No. 4,344,949), ramipril (U.S. Pat. No. 4,587,258), and perindopril (U.S. Pat. No. 4,508,729), carboxyalkyl dipeptide mimics such as cilazapril (U.S. Pat. No. 4,512,924) and benazapril (U.S. Pat. No. 4,410,520), phosphinylalkanoyl prolines such as fosinopril (U.S. Pat. No. 4,337,201) and trandolopril.

Renin inhibitors are compounds which interfere with the activity of renin. Renin inhibitors include amino acids and derivatives thereof, peptides and derivatives thereof, and antibodies to renin. Examples of renin inhibitors that are the subject of United States patents are as follows: urea derivatives of peptides (U.S. Pat. No. 5,116,835); amino acids connected by nonpeptide bonds (U.S. Pat. No. 5,114,937); di- and tri-peptide derivatives (U.S. Pat. No. 5,106,835); amino acids and derivatives thereof (U.S. Pat. Nos. 5,104,869 and 5,095,119); diol sulfonamides and sulfinyls (U.S. Pat. No. 5,098,924); modified peptides (U.S. Pat. No. 5,095,006); peptidyl beta-aminoacyl aminodiol carbamates (U.S. Pat. No. 5,089,471); pyrolimidazolones (U.S. Pat. No. 5,075,451); fluorine and chlorine statine or statone containing peptides (U.S. Pat. No. 5,066,643); peptidyl amino diols (U.S. Pat. Nos. 5,063,208 and 4,845,079); N-morpholino derivatives (U.S. Pat. No. 5,055,466); pepstatin derivatives (U.S. Pat. No. 4,980,283); N-heterocyclic alcohols (U.S. Pat. No. 4,885,292); monoclonal antibodies to renin (U.S. Pat. No. 4,780,401); and a variety of other peptides and analogs thereof (U.S. Pat. Nos. 5,071,837, 5,064,965, 5,063,207, 5,036,054, 5,036,053, 5,034,512, and 4,894,437).

HMG-CoA reductase inhibitors include, but are not limited to, statins such as simvastatin (U.S. Pat. No. 4,444,784), lovastatin (U.S. Pat. No. 4,231,938), pravastatin sodium (U.S. Pat. No. 4,346,227), fluvastatin (U.S. Pat. No. 4,739,073), atorvastatin (U.S. Pat. No. 5,273,995), cerivastatin, and numerous others described in U.S. Pat. No. 5,622,985, U.S. Pat. No. 5,135,935, U.S. Pat. No. 5,356,896, U.S. Pat. No. 4,920,109, U.S. Pat. No. 5,286,895, U.S. Pat. No. 5,262,435, U.S. Pat. No. 5,260,332, U.S. Pat. No. 5,317,031, U.S. Pat. No. 5,283,256, U.S. Pat. No. 5,256,689, U.S. Pat. No. 5,182,298, U.S. Pat. No. 5,369,125, U.S. Pat. No. 5,302,604, U.S. Pat. No. 5,166,171, U.S. Pat. No. 5,202,327, U.S. Pat. No. 5,276,021, U.S. Pat. No. 5,196,440, U.S. Pat. No. 5,091,386, U.S. Pat. No. 5,091,378, U.S. Pat. No. 4,904,646, U.S. Pat. No. 5,385,932, U.S. Pat. No. 5,250,435, U.S. Pat. No. 5,132,312, U.S. Pat. No. 5,130,306, U.S. Pat. No. 5,116,870, U.S. Pat. No. 5,112,857, U.S. Pat. No. 5,102,911, U.S. Pat. No. 5,098,931, U.S. Pat. No. 5,081,136, U.S. Pat. No. 5,025,000, U.S. Pat. No. 5,021,453, U.S. Pat. No. 5,017,716, U.S. Pat. No. 5,001,144, U.S. Pat. No. 5,001,128, U.S. Pat. No. 4,997,837, U.S. Pat. No. 4,996,234, U.S. Pat. No. 4,994,494, U.S. Pat. No. 4,992,429, U.S. Pat. No. 4,970,231, U.S. Pat. No. 4,968,693, U.S. Pat. No. 4,963,538, U.S. Pat. No. 4,957,940, U.S. Pat. No. 4,950,675, U.S. Pat. No. 4,946,864, U.S. Pat. No. 4,946,860, U.S. Pat. No. 4,940,800, U.S. Pat. No. 4,940,727, U.S. Pat. No. 4,939,143, U.S. Pat. No. 4,929,620, U.S. Pat. No. 4,923,861, U.S. Pat. No. 4,906,657, U.S. Pat. No. 4,906,624 and U.S. Pat. No. 4,897,402, the disclosures of which patents are incorporated herein by reference.

It is to be understood that the invention contemplates the use of one or more of any of the foregoing agents in combination with use of miRNA-144 as a companion diagnostic and/or as a prophylactic and/or therapeutic agent itself.

### Pharmaceutical Compositions

miRNA-144 may be used (e.g., administered) in pharmaceutically acceptable preparations (or pharmaceutically acceptable compositions), typically when combined with a pharmaceutically acceptable carrier. Such preparations may routinely contain pharmaceutically acceptable concentrations of salt, buffering agents, preservatives, compatible carriers, and may optionally comprise other (i.e., secondary) therapeutic agents, as discussed above.

A pharmaceutically acceptable carrier is a pharmaceutically acceptable material, composition or vehicle, such as a liquid or solid filler, diluent, excipient, solvent or encapsulating material, involved in carrying or transporting a prophylactically or therapeutically active agent. Each carrier must be “acceptable” in the sense of being compatible with the other ingredients of the formulation and not injurious to the subject. Some examples of materials which can serve as pharmaceutically acceptable carriers include sugars, such as lactose, glucose and sucrose; glycols, such as propylene glycol; polyols, such as glycerin, sorbitol, mannitol and polyethylene glycol; esters, such as ethyl oleate and ethyl laurate; buffering agents, such as magnesium hydroxide and aluminum hydroxide; pyrogen-free water; isotonic saline; Ringer's solution; ethyl alcohol; phosphate buffer solutions; and other non-toxic compatible substances employed in pharmaceutical formulations.

The agents, when it is desirable to deliver them systemically, may be formulated for parenteral administration by injection, including for example by bolus injection or continuous infusion. Formulations for injection may be presented in unit dosage form, e.g., in ampoules or in multi-dose containers, with or without an added preservative.

The compositions may take such forms as water-soluble suspensions, solutions or emulsions in oily or aqueous vehicles, and may contain formulatory agents such as suspending, stabilizing and/or dispersing agents. Suitable lipophilic solvents or vehicles include fatty oils such as sesame oil, or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or agents which increase solubility. Alternatively, the agents may be in lyophilized or other powder or solid form for constitution with a suitable vehicle, e.g., sterile pyrogen-free water, before use.

Pharmaceutical compositions of the invention formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition of the invention. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) and as much as 100% (w/w) of the active ingredient, and may comprise one or more of the additional ingredients described herein. A pharmaceutical composition of the invention can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Other delivery systems can include time-release, delayed release or sustained release delivery systems. Such systems can avoid repeated administrations of the active compound, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189 and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be desirable. Long-term release, are used herein, means that the implant is constructed and arranged to delivery therapeutic levels of the active ingredient for at least 30 days, and preferably 60 days. Long-term sustained release implants are known to those of ordinary skill in the art and include some of the release systems described above.

In addition, nucleic acids may be directly administered to the subject or may be administered in conjunction with a nucleic acid delivery complex. A nucleic acid delivery complex shall mean a nucleic acid molecule associated with (e.g. ionically or covalently bound to; or encapsulated within) a targeting means (e.g. a molecule that results in higher affinity binding to target cell and/or increased cellular uptake by target cells). Examples of nucleic acid delivery complexes include nucleic acids associated with a sterol (e.g. cholesterol), a lipid (e.g. a cationic lipid, virosome or liposome), or a target cell specific binding agent (e.g. a ligand recognized by target cell specific receptor). Preferred complexes may be sufficiently stable in vivo to prevent significant uncoupling prior to internalization by the target cell. However, the complex can be cleavable under appropriate conditions within the cell so that the nucleic acid is released in a functional form.

Delivery vehicles or delivery devices for delivering nucleic acids to surfaces have been described. The nucleic acid and/or other therapeutics may be administered alone (e.g., in saline or buffer) or using any delivery vehicles known in the art. For instance the following delivery vehicles have been described: Cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); Liposomes (Childers et al., 1999, Michalek et al., 1989, 1992, de Haan 1995a, 1995b); Live bacterial vectors (e.g., Salmonella, Escherichia coli, Bacillus calmatte-guerin, Shigella, Lactobacillus) (Hone et al., 1996, Pouwels et al., 1998, Chatfield et al., 1993, Stover et al., 1991, Nugent et al., 1998); Live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); Microspheres (Gupta et al., 1998, Jones et al., 1996, Maloy et al., 1994, Moore et al., 1995, O'Hagan et al., 1994, Eldridge et al., 1989); Nucleic acid vaccines (Fynan et al., 1993, Kuklin et al., 1997, Sasaki et al., 1998, Okada et al., 1997, Ishii et al., 1997); Polymers (e.g. carboxymethylcellulose, chitosan) (Hamajima et al., 1998, Jabbal-Gill et al., 1998); Polymer rings (Wyatt et al., 1998); Proteosomes (Vancott et al., 1998, Lowell et al., 1988, 1996, 1997); Sodium Fluoride (Hashi et al., 1998); Transgenic plants (Tacket et al., 1998, Mason et al., 1998, Haq et al., 1995); Virosomes (Gluck et al., 1992, Mengiardi et al., 1995, Cryz et al., 1998); Virus-like particles (Jiang et al., 1999, Leibl et al., 1998). Other delivery vehicles are known in the art and some additional examples are provided below in the discussion of vectors.

### Effective Amounts

The preparations of the invention are administered in effective amounts. An effective amount is that amount of an agent that alone stimulates the desired outcome. The absolute amount will depend upon a variety of factors, including the material selected for administration, whether the administration is in single or multiple doses, and individual patient parameters including age, physical condition, size, weight, and the stage of the disease. These factors are well known to those of ordinary skill in the art and can be addressed with no more than routine experimentation.

The exact amount of the agent (e.g., the miRNA-144 therapy) required to achieve an effective amount will vary from subject to subject, depending, for example, on species, age, and general condition of a subject, severity of the side effects or disorder, identity of the particular compound, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of the agent or a preparation thereof for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a molecule per unit dosage form.

In certain embodiments, the agents may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

### Administration Routes

The compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration).

### Nucleic Acids and Derivatives Thereof

The term “nucleic acid” refers to multiple linked nucleotides (i.e., molecules comprising a sugar (e.g., ribose or deoxyribose) linked to an exchangeable organic base, which is either a pyrimidine (e.g., cytosine (C), thymidine (T) or uracil (U)) or a purine (e.g., adenine (A) or guanine (G)). “Nucleic acid” and “nucleic acid molecule” are used interchangeably and refer to oligoribonucleotides as well as oligodeoxyribonucleotides. The terms shall also include polynucleosides (i.e., a polynucleotide minus a phosphate) and any other organic base containing nucleic acid. The organic bases include adenine, uracil, guanine, thymine, cytosine and inosine. The nucleic acids may be single- or double-stranded. Nucleic acids can be obtained from natural sources, or can be synthesized using a nucleic acid synthesizer.

As used herein with respect to linked units of a nucleic acid, “linked” or “linkage” means two entities bound to one another by any physicochemical means. Any linkage known to those of ordinary skill in the art, covalent or non-covalent, is embraced. Natural linkages, which are those ordinarily found in nature connecting for example the individual units of a particular nucleic acid, are most common. Natural linkages include, for instance, amide, ester and thioester linkages. The individual units of a nucleic acid may be linked, however, by synthetic or modified linkages. Nucleic acids where the units are linked by covalent bonds will be most common but those that include hydrogen bonded units are also embraced by the invention. It is to be understood that all possibilities regarding nucleic acids apply equally to nucleic acid tails, nucleic acid probes and capture nucleic acids.

The terms “nucleic acid” and “oligonucleotide” are used interchangeably herein to mean multiple nucleotides (i.e. molecules comprising a sugar (e.g. ribose or deoxyribose) linked to a phosphate group and to an exchangeable organic base, which is either a substituted pyrimidine (e.g. cytosine (C), thymidine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acid molecules can be obtained from existing nucleic acid sources (e.g., genomic or cDNA), but are preferably synthetic (e.g. produced by nucleic acid synthesis).

Nucleic acids can encompass various chemical modifications and substitutions, in comparison to natural RNA and DNA, involving a phosphodiester internucleoside bridge, a β-D-ribose unit and/or a natural nucleoside base (adenine, guanine, cytosine, thymine, uracil). Examples of chemical modifications are known to the skilled person and are described, for example, in Uhlmann E et al. (1990) Chem Rev 90:543; “Protocols for Oligonucleotides and Analogs” Synthesis and Properties & Synthesis and Analytical Techniques, S. Agrawal, Ed, Humana Press, Totowa, USA 1993; Crooke S T et al. (1996) Annu Rev Pharmacol Toxicol 36:107-129; and Hunziker J et al. (1995) Mod Synth Methods 7:331-417. A nucleic acid may have one or more modifications (for example relative to naturally occurring nucleic acids), wherein each modification is located at a particular phosphodiester internucleoside bridge and/or at a particular β-D-ribose unit and/or at a particular natural nucleoside base position in comparison to an oligonucleotide of the same sequence which is composed of natural DNA or RNA.

For example, the oligonucleotides may comprise one or more modifications and wherein each modification is independently selected from:
a) the replacement of a phosphodiester internucleoside bridge located at the 3′ and/or the 5′ end of a nucleoside by a modified internucleoside bridge,
b) the replacement of phosphodiester bridge located at the 3′ and/or the 5′ end of a nucleoside by a dephospho bridge,
c) the replacement of a sugar phosphate unit from the sugar phosphate backbone by another unit,
d) the replacement of a β-D-ribose unit by a modified sugar unit, and
e) the replacement of a natural nucleoside base by a modified nucleoside base.

More detailed examples for the chemical modification of an oligonucleotide are as follows.

Nucleic acids also include substituted purines and pyrimidines such as C-5 propyne pyrimidine and 7-deaza-7-substituted purine modified bases. Wagner R W et al. (1996) Nat Biotechnol 14:840-4. Purines and pyrimidines include but are not limited to adenine, cytosine, guanine, thymidine, 5-methylcytosine, 2-aminopurine, 2-amino-6-chloropurine, 2,6-diaminopurine, hypoxanthine, and other naturally and non-naturally occurring nucleobases, substituted and unsubstituted aromatic moieties. Other such modifications are well known to those of skill in the art. In all of the foregoing embodiments, an X residue can also be a non-naturally occurring nucleotide, or a nucleotide analog, such as those described herein.

A modified base is any base which is chemically distinct from the naturally occurring bases typically found in DNA and RNA such as T, C, G, A, and U, but which share basic chemical structures with these naturally occurring bases. The modified nucleoside base may be, for example, selected from hypoxanthine, uracil, dihydrouracil, pseudouracil, 2-thiouracil, 4-thiouracil, 5-aminouracil, 5-(C₁-C₆)-alkyluracil, 5-(C₂-C₆)-alkenyluracil, 5-(C₂-C₆)-alkynyluracil, 5-(hydroxymethyl)uracil, 5-chlorouracil, 5-fluorouracil, 5-bromouracil, 5-hydroxycytosine, 5-(C₁-C₆)-alkylcytosine, 5-(C₂-C₆)-alkenylcytosine, 5-(C₂-C₆)-alkynylcytosine, 5-chlorocytosine, 5-fluorocytosine, 5-bromocytosine, N²-dimethylguanine, 2,4-diamino-purine, 8-azapurine, a substituted 7-deazapurine, preferably 7-deaza-7-substituted and/or 7-deaza-8-substituted purine, 5-hydroxymethylcytosine, N4-alkylcytosine, e.g., N4-ethylcytosine, 5-hydroxydeoxycytidine, 5-hydroxymethyldeoxycytidine, N4-alkyldeoxycytidine, e.g., N4-ethyldeoxycytidine, 6-thiodeoxyguanosine, and deoxyribonucleosides of nitropyrrole, C5-propynylpyrimidine, and diaminopurine e.g., 2,6-diaminopurine, inosine, 5-methylcytosine, 2-aminopurine, 2-amino-6-chloropurine, hypoxanthine or other modifications of a natural nucleoside bases. This list is meant to be exemplary and is not to be interpreted to be limiting.

A modified cytosine as used herein is a naturally occurring or non-naturally occurring pyrimidine base analog of cytosine which can replace this base without impairing the immunostimulatory activity of the oligonucleotide. Modified cytosines include but are not limited to 5-substituted cytosines (e.g. 5-methyl-cytosine, 5-fluoro-cytosine, 5-chloro-cytosine, 5-bromo-cytosine, 5-iodo-cytosine, 5-hydroxy-cytosine, 5-hydroxymethyl-cytosine, 5-difluoromethyl-cytosine, and unsubstituted or substituted 5-alkynyl-cytosine), 6-substituted cytosines, N4-substituted cytosines (e.g. N4-ethyl-cytosine), 5-aza-cytosine, 2-mercapto-cytosine, isocytosine, pseudo-isocytosine, cytosine analogs with condensed ring systems (e.g. N,N′-propylene cytosine or phenoxazine), and uracil and its derivatives (e.g. 5-fluoro-uracil, 5-bromo-uracil, 5-bromovinyl-uracil, 4-thio-uracil, 5-hydroxy-uracil, 5-propynyl-uracil). Some of the preferred cytosines include 5-methyl-cytosine, 5-fluoro-cytosine, 5-hydroxy-cytosine, 5-hydroxymethyl-cytosine, and N4-ethyl-cytosine. A cytosine base may be substituted by a universal base (e.g. 3-nitropyrrole, P-base), an aromatic ring system (e.g. fluorobenzene or difluorobenzene) or a hydrogen atom (dSpacer).

A modified guanine as used herein is a naturally occurring or non-naturally occurring purine base analog of guanine which can replace this base without impairing the immunostimulatory activity of the oligonucleotide. Modified guanines include but are not limited to 7-deazaguanine, 7-deaza-7-substituted guanine (such as 7-deaza-7-(C2-C6)alkynylguanine), 7-deaza-8-substituted guanine, hypoxanthine, N2-substituted guanines (e.g. N2-methyl-guanine), 5-amino-3-methyl-3H,6H-thiazolo[4,5-d]pyrimidine-2,7-dione, 2,6-diaminopurine, 2-aminopurine, purine, indole, adenine, substituted adenines (e.g. N6-methyl-adenine, 8-oxo-adenine) 8-substituted guanine (e.g. 8-hydroxyguanine and 8-bromoguanine), and 6-thioguanine. The guanine base may be substituted by a universal base (e.g. 4-methyl-indole, 5-nitro-indole, and K-base), an aromatic ring system (e.g. benzimidazole or dichloro-benzimidazole, 1-methyl-1H-[1,2,4]triazole-3-carboxylic acid amide) or a hydrogen atom (dSpacer).

The nucleic acids may include modified internucleotide linkages, such as those described in above. These modified linkages may be partially resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length or secondary structure. Nucleic acids that are tens to hundreds of kilobases long are relatively resistant to in vivo degradation. For shorter nucleic acids, secondary structure can stabilize and increase their effect. For example, if the 3′ end of an nucleic acid has self-complementarity to an upstream region, so that it can fold back and form a sort of stem loop structure, then the nucleic acid becomes stabilized and therefore exhibits more activity.

Nucleic acid stabilization can also be accomplished via phosphate backbone modifications. Oligonucleotides having phosphorothioate linkages, in some embodiments, may provide maximal activity and protect the oligonucleotide from degradation by intracellular exo- and endo-nucleases.

Modification of the nucleic acid backbone may provide enhanced activity of nucleic acids when administered in vivo, at least as a result of a longer half-life in vivo, enhanced nuclease resistance, increased cellular uptake, increased protein binding, and/or altered intracellular localization. Constructs having phosphorothioate linkages provide maximal activity and protect the nucleic acid from degradation by intracellular exo- and endo-nucleases. Other modified nucleic acids include phosphodiester modified nucleic acids, combinations of phosphodiester and phosphorothioate nucleic acid, methylphosphonate, methylphosphorothioate, phosphorodithioate, p-ethoxy, and combinations thereof.

Other stabilized nucleic acids include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acids which contain diol, such as tetraethyleneglycol or hexaethyleneglycol, at either or both termini have also been shown to be substantially resistant to nuclease degradation.

The nucleic acids may have one or two accessible 5′ ends. It is possible to create modified oligonucleotides having two such 5′ ends, for instance, by attaching two oligonucleotides through a 3′-3′ linkage to generate an oligonucleotide having one or two accessible 5′ ends. The 3′3′-linkage may be a phosphodiester, phosphorothioate or any other modified internucleoside bridge. Methods for accomplishing such linkages are known in the art. For instance, such linkages have been described in Seliger, H. et al., Oligonucleotide analogs with terminal 3′-3′- and 5′-5′-internucleotidic linkages as antisense inhibitors of viral gene expression, Nucleosides & Nucleotides (1991), 10(1-3), 469-77 and Jiang, et al., Pseudo-cyclic oligonucleotides: in vitro and in vivo properties, Bioorganic & Medicinal Chemistry (1999), 7(12), 2727-2735.

Additionally, 3′3′-linked ODNs where the linkage between the 3′-terminal nucleosides is not a phosphodiester, phosphorothioate or other modified bridge, can be prepared using an additional spacer, such as tri- or tetra-ethylenglycol phosphate moiety (Durand, M. et al, Triple-helix formation by an oligonucleotide containing one (dA)12 and two (dT)12 sequences bridged by two hexaethylene glycol chains, Biochemistry (1992), 31(38), 9197-204, U.S. Pat. No. 5,658,738, and U.S. Pat. No. 5,668,265). Alternatively, the non-nucleotidic linker may be derived from ethanediol, propanediol, or from an abasic deoxyribose (dSpacer) unit (Fontanel, Marie Laurence et al., Sterical recognition by T4 polynucleotide kinase of non-nucleosidic moieties 5′-attached to oligonucleotides; Nucleic Acids Research (1994), 22(11), 2022-7) using standard phosphoramidite chemistry. The non-nucleotidic linkers can be incorporated once or multiple times, or combined with each other allowing for any desirable distance between the 3′-ends of the two nucleic acids to be linked.

A phosphodiester internucleoside bridge located at the 3′ and/or the 5′ end of a nucleoside can be replaced by a modified internucleoside bridge, wherein the modified internucleoside bridge is for example selected from phosphorothioate, phosphorodithioate, NR¹R²-phosphoramidate, boranophosphate, α-hydroxybenzyl phosphonate, phosphate-(C₁-C₂₁)—O-alkyl ester, phosphate-[(C₆-C₁₂)aryl-(C₁-C₂₁)—O-alkyl]ester, (C₁-C₈)alkylphosphonate and/or (C₆-C₁₂)arylphosphonate bridges, (C₇-C₁₂)-α-hydroxymethyl-aryl (e.g., disclosed in WO 95/01363), wherein (C₆-C₁₂)aryl, (C₆-C₂₀)aryl and (C₆-C₁₄)aryl are optionally substituted by halogen, alkyl, alkoxy, nitro, cyano, and where R¹ and R² are, independently of each other, hydrogen, (C₁-C₁₈)-alkyl, (C₆-C₂₀)-aryl, (C₆-C₁₄)-aryl-(C₁-C₈)-alkyl, preferably hydrogen, (C₁-C₈)-alkyl, preferably (C₁-C₄)-alkyl and/or methoxyethyl, or R¹ and R² form, together with the nitrogen atom carrying them, a 5-6-membered heterocyclic ring which can additionally contain a further heteroatom from the group 0, S and N.

The replacement of a phosphodiester bridge located at the 3′ and/or the 5′ end of a nucleoside by a dephospho bridge (dephospho bridges are described, for example, in Uhlmann E and Peyman A in “Methods in Molecular Biology”, Vol. 20, “Protocols for Oligonucleotides and Analogs”, S. Agrawal, Ed., Humana Press, Totowa 1993, Chapter 16, pp. 355 ff), wherein a dephospho bridge is for example selected from the dephospho bridges formacetal, 3′-thioformacetal, methylhydroxylamine, oxime, methylenedimethyl-hydrazo, dimethylenesulfone and/or silyl groups.

The nucleic acids may have chimeric backbones. As used herein, a chimeric backbone is one that comprises more than one type of linkage. In some embodiments, phosphorothioate linkages may be present at the 5′ and 3′ termini of the nucleic acid, and the remainder of the linkages may be phosphodiester. The termini may comprise one, two or more non-phosphodiester linkages.

The nucleic acids also include nucleic acids having backbone sugars which are covalently attached to low molecular weight organic groups other than a hydroxyl group at the 2′ position and other than a phosphate group at the 5′ position. Thus, modified nucleic acids may include a 2′-O-alkylated ribose group. In addition, modified nucleic acids may include sugars such as arabinose or 2′-fluoroarabinose instead of ribose. Thus the nucleic acids may be heterogeneous in backbone composition thereby containing any possible combination of polymer units linked together such as peptide-nucleic acids (which have amino acid backbone with nucleic acid bases). In some embodiments, the nucleic acids are homogeneous in backbone composition. Other examples are described in more detail below.

A sugar phosphate unit (i.e., a β-D-ribose and phosphodiester internucleoside bridge together forming a sugar phosphate unit) from the sugar phosphate backbone (i.e., a sugar phosphate backbone is composed of sugar phosphate units) can be replaced by another unit, wherein the other unit is for example suitable to build up a “morpholino-derivative” oligomer (as described, for example, in Stirchak E P et al. (1989) Nucleic Acids Res 17:6129-41), that is, e.g., the replacement by a morpholino-derivative unit; or to build up a polyamide nucleic acid (“PNA”; as described for example, in Nielsen P E et al. (1994) Bioconjug Chem 5:3-7), that is, e.g., the replacement by a PNA backbone unit, e.g., by 2-aminoethylglycine. The oligonucleotide may have other carbohydrate backbone modifications and replacements, such as peptide nucleic acids with phosphate groups (PHONA), locked nucleic acids (LNA), and oligonucleotides having backbone sections with alkyl linkers or amino linkers. The alkyl linker may be branched or unbranched, substituted or unsubstituted, and chirally pure or a racemic mixture.

A β-ribose unit or a β-D-2′-deoxyribose unit can be replaced by a modified sugar unit, wherein the modified sugar unit is for example selected from β-D-ribose, α-D-2′-deoxyribose, L-2′-deoxyribose, 2′-F-2′-deoxyribose, 2′-F-arabinose, 2′-O—(C₁-C₆)alkyl-ribose, preferably 2′-O—(C₁-C₆)alkyl-ribose is 2′-O-methylribose, 2′-O—(C₂-C₆)alkenyl-ribose, 2′-[O—(C₁-C₆)alkyl-O—(C₁-C₆)alkyl]-ribose, 2′-NH₂-2′-deoxyribose, β-D-xylo-furanose, α-arabinofuranose, 2,4-dideoxy-β-D-erythro-hexo-pyranose, and carbocyclic (described, for example, in Froehler J (1992) Am Chem Soc 114:8320) and/or open-chain sugar analogs (described, for example, in Vandendriessche et al. (1993) Tetrahedron 49:7223) and/or bicyclosugar analogs (described, for example, in Tarkov M et al. (1993) Helv Chim Acta 76:481).

In some embodiments the sugar is 2′-O-methylribose, particularly for one or both nucleotides linked by a phosphodiester or phosphodiester-like internucleoside linkage.

In some embodiments, the nucleic acids may include a peptide nucleic acid (PNA), a bisPNA clamp, a pseudocomplementary PNA, a locked nucleic acid (LNA), DNA, RNA, or co-nucleic acids of the above such as DNA-LNA co-nucleic acids (as described in co-pending U.S. patent application Ser. No. 10/421,644 and publication number US 2003-0215864 A1 and published Nov. 20, 2003, and PCT application having serial number PCT/US03/12480 and publication number WO 03/091455 A1 and published Nov. 6, 2003, filed on Apr. 23, 2003), or co-polymers thereof (e.g., a DNA-LNA co-polymer).

For use in the instant invention, the nucleic acids of the invention can be synthesized de novo using any of a number of procedures well known in the art. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated nucleic acid synthesizers available in the market. These oligonucleotides are referred to as synthetic oligonucleotides.

Modified backbones such as phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (e.g., Uhlmann, E. and Peyman, A., Chem. Rev. 90:544, 1990; Goodchild, J., Bioconjugate Chem. 1:165, 1990).

Nucleic acids prepared in this manner are referred to as isolated nucleic acid. An “isolated nucleic acid” generally refers to a nucleic acid which is separated from components with which it is normally associated (including for example a natural environment (e.g., a cell) or an in vitro environment (e.g., a biochemical reaction mixture).

It is to be understood that the foregoing discussion of oligonucleotides and nucleic acids applies to the nucleic acids of the invention that are used for detection purposes (e.g., probes) and those that are used for in vivo purposes (e.g., as therapy).

### Detectable Labeling of Nucleic Acids

Nucleic acids such as nucleic acid probes may be detectably labeled (i.e., they may comprise a detectable label). A detectable label is a moiety, the presence of which can be ascertained directly or indirectly. Generally, detection of the label involves the creation of a detectable signal such as for example an emission of energy. The label may be of a chemical, lipid, peptide or nucleic acid nature although it is not so limited. The nature of label used will depend on a variety of factors, including the nature of the analysis being conducted, the type of the energy source and detector used. The label should be sterically and chemically compatible with the constituents to which it is bound.

The label can be detected directly for example by its ability to emit and/or absorb electromagnetic radiation of a particular wavelength. A label can be detected indirectly for example by its ability to bind, recruit and, in some cases, cleave another moiety which itself may emit or absorb light of a particular wavelength (e.g., an epitope tag such as the FLAG epitope, an enzyme tag such as horseradish peroxidase, etc.).

There are several known methods of direct chemical labeling of DNA. (Hermanson, G. T., Bioconjugate Techniques, Academic Press, Inc., San Diego, 1996; Roget et al., 1989; Proudnikov and Mirabekov, Nucleic Acid Research, 24:4535-4532, 1996.) One of the methods is based on the introduction of aldehyde groups by partial depurination of DNA. Fluorescent labels with an attached hydrazine group are efficiently coupled with the aldehyde groups and the hydrazine bonds are stabilized by reduction with sodium labeling efficiencies around 60%. The reaction of cytosine with bisulfite in the presence of an excess of an amine fluorophore leads to transamination at the N4 position (Hermanson, 1996). Reaction conditions such as pH, amine fluorophore concentration, and incubation time and temperature affect the yield of products formed. At high concentrations of the amine fluorophore (3M), transamination can approach 100% (Draper and Gold, 1980).

It is also possible to synthesize nucleic acids de novo (e.g., using automated nucleic acid synthesizers) using fluorescently labeled nucleotides. Such nucleotides are commercially available from suppliers such as Amersham Pharmacia Biotech, Molecular Probes, and New England Nuclear/Perkin Elmer.

Generally the detectable label can be selected from the group consisting of directly detectable labels such as a fluorescent molecule (e.g., fluorescein, rhodamine, tetramethylrhodamine, R-phycoerythrin, Cy-3, Cy-5, Cy-7, Texas Red, Phar-Red, allophycocyanin (APC), fluorescein amine, eosin, dansyl, umbelliferone, 5-carboxyfluorescein (FAM), 2′7′-dimethoxy-4′5′-dichloro-6-carboxyfluorescein (JOE), 6 carboxyrhodamine (R6G), N,N,N′,N′-tetramethyl-6-carboxyrhodamine (TAMRA), 6-carboxy-X-rhodamine (ROX), 4-(4′-dimethylaminophenylazo)benzoic acid (DABCYL), 5-(2′-aminoethyl)aminonaphthalene-1-sulfonic acid (EDANS), 4-acetamido-4′-isothiocyanatostilbene-2,2′disulfonic acid, acridine, acridine isothiocyanate, r-amino-N-(3-vinylsulfonyl)phenylnaphthalimide-3,5, disulfonate (Lucifer Yellow VS), N-(4-anilino-1-naphthyl)maleimide, anthranilamide, Brilliant Yellow, coumarin, 7-amino-4-methylcoumarin, 7-amino-4-trifluoromethylcouluarin (Coumarin 151), cyanosine, 4′,6-diaminidino-2-phenylindole (DAPI), 5′,5″-diaminidino-2-phenylindole (DAPI), 5′,5″-dibromopyrogallol-sulfonephthalein (Bromopyrogallol Red), 7-diethylamino-3-(4′-isothiocyanatophenyl)-4-methylcoumarin diethylenetriamine pentaacetate, 4,4′-diisothiocyanatodihydro-stilbene-2,2′-disulfonic acid, 4,4′-diisothiocyanatostilbene-2,2′-disulfonic acid, 4-dimethylaminophenylazophenyl-4′-isothiocyanate (DABITC), eosin isothiocyanate, erythrosin B, erythrosin isothiocyanate, ethidium, 5-(4,6-dichlorotriazin-2-yl)aminofluorescein (DTAF), QFITC (XRITC), fluorescamine, IR144, IR1446, Malachite Green isothiocyanate, 4-methylumbelliferone, ortho cresolphthalein, nitrotyrosine, pararosaniline, Phenol Red, B-phycoerythrin, o-phthaldialdehyde, pyrene, pyrene butyrate, succinimidyl 1-pyrene butyrate, Reactive Red 4 (Cibacron® Brilliant Red 3B-A), lissamine rhodamine B sulfonyl chloride, rhodamine B, rhodamine 123, rhodamine X, sulforhodamine B, sulforhodamine 101, sulfonyl chloride derivative of sulforhodamine 101, tetramethyl rhodamine, riboflavin, rosolic acid, and terbium chelate derivatives), a chemiluminescent molecule, a bioluminescent molecule, a chromogenic molecule, a radioisotope (e.g., P32 or H3, 14C, 125I and 131I), an electron spin resonance molecule (such as for example nitroxyl radicals), an optical or electron density molecule, an electrical charge transducing or transferring molecule, an electromagnetic molecule such as a magnetic or paramagnetic bead or particle, a semiconductor nanocrystal or nanoparticle (such as quantum dots described for example in U.S. Pat. No. 6,207,392 and commercially available from Quantum Dot Corporation and Evident Technologies), a colloidal metal, a colloid gold nanocrystal, a nuclear magnetic resonance molecule, and the like.

The detectable label can also be selected from the group consisting of indirectly detectable labels such as an enzyme (e.g., alkaline phosphatase, horseradish peroxidase, -galactosidase, glucoamylase, lysozyme, luciferases such as firefly luciferase and bacterial luciferase (U.S. Pat. No. 4,737,456); saccharide oxidases such as glucose oxidase, galactose oxidase, and glucose-6-phosphate dehydrogenase; heterocyclic oxidases such as uricase and xanthine oxidase coupled to an enzyme that uses hydrogen peroxide to oxidize a dye precursor such as HRP, lactoperoxidase, or microperoxidase), an enzyme substrate, an affinity molecule, a ligand, a receptor, a biotin molecule, an avidin molecule, a streptavidin molecule, an antigen (e.g., epitope tags such as the FLAG or HA epitope), a hapten (e.g., biotin, pyridoxal, digoxigenin fluorescein and dinitrophenol), an antibody, an antibody fragment, a microbead, and the like. Antibody fragments include Fab, F(ab)2, Fd and antibody fragments which include a CDR3 region.

In some embodiments, the first and second probes may be labeled with fluorophores that form a fluorescence resonance energy transfer (FRET) pair. In this case, one excitation wavelength is used to excite fluorescence of donor fluorophores. A portion of the energy absorbed by the donors can be transferred to acceptor fluorophores if they are close enough spatially to the donor molecules (i.e., the distance between them must approximate or be less than the Forster radius or the energy transfer radius). Once the acceptor fluorophore absorbs the energy, it in turn fluoresces in its characteristic emission wavelength. Since energy transfer is possible only when the acceptor and donor are located in close proximity, acceptor fluorescence is unlikely if both probes are not bound to the same miRNA. Acceptor fluorescence therefore can be used to determine presence of miRNA.

It is to be understood however that if a FRET fluorophore pair is used, coincident binding of the pair to a single target is detected by the presence or absence of a signal rather than a coincident detection of two signals.

A FRET fluorophore pair is two fluorophores that are capable of undergoing FRET to produce or eliminate a detectable signal when positioned in proximity to one another. Examples of donors include Alexa 488, Alexa 546, BODIPY 493, Oyster 556, Fluor (FAM), Cy3 and TMR (Tamra). Examples of acceptors include Cy5, Alexa 594, Alexa 647 and Oyster 656. Cy5 can work as a donor with Cy3, TMR or Alexa 546, as an example. FRET should be possible with any fluorophore pair having fluorescence maxima spaced at 50-100 nm from each other. The FRET embodiment can be coupled with another label on the target miRNA such as a backbone label, as discussed below.

### Kits

The invention contemplates a variety of kits. Some aspects of the invention contemplate a kit to be used to measure miRNA-144 levels in a biological sample from a subject. Some kits can be used to measure one or more miRNAs including but not limited to miRNA-144. In certain embodiments, a kit contains miRNA probes, synthetic miRNA molecules or miRNA inhibitors, or any range and combination derivable therein. Such kits may comprise in suitable container means, one or more miRNA probes and/or amplification primers, wherein the miRNA probes detect or primer amplify one or more miRNA described herein. The kit can further comprise reagents for labeling miRNA in the sample. The kit may also include the labeling reagents include at least one amine-modified nucleotide, poly(A) polymerase, and poly(A) polymerase buffer. Labeling reagents can include an amine-reactive dye.

Some aspects of the invention contemplate a kit to be used to administer miRNA-144 therapy to a subject in need thereof. Some aspects of the invention contemplate a kit to be used to do both. Such aspects encompass in some instances a packaged and labeled miRNA-144 based pharmaceutical product. This article of manufacture or kit may include the appropriate unit dosage form in an appropriate vessel or container such as a glass vial or plastic ampoule or other container that is hermetically sealed. Preferably, the article of manufacture or kit further comprises instructions on how to use including how to administer the pharmaceutical product. The instructions may further contain informational material that advises a medical practitioner, technician or subject on how to appropriately prevent or treat the disease or disorder in question. In other words, the article of manufacture includes instructions indicating or suggesting a dosing regimen for use including but not limited to actual doses, monitoring procedures, and other monitoring information.

As with any pharmaceutical product, the packaging material and container are designed to protect the stability of the product during storage and shipment.

The kits may include agents in sterile aqueous suspensions that may be used directly or may be diluted with normal saline for intravenous injection or use in a nebulizer, or dilution or combination with surfactant for intratracheal administration. The kits may therefore also contain the diluent solution or agent, such as saline or surfactant. The kit may also include a pulmonary delivery device such as a nebulizer or disposable components therefore such as the mouthpiece, nosepiece, or mask.

### EXAMPLES

We have shown that remote IC induces changes in gene expression in mouse myocardium and human neutrophils. Small noncoding microRNAs (miRNAs or miRs) modify post-transcriptional protein expression and gene expression, and thus may participate in the cardioprotection induced by remote IC. Using miRNA array analysis, we examined miRNA signatures in mouse heart after remote IC. We found remote IC up-regulated miR-144 in a manner consistent with its cardioprotective responses to ischemia-reperfusion injury. These results supplement our understanding of the cell signaling mechanisms underlying cardioprotection.

### Materials and Methods Generally:

The following materials and methods were used in the following Examples unless otherwise indicated.

Animals and Subjects:

All animal protocols were approved by the Animal Care and Use Committee of the Hospital for Sick Children in Toronto and conformed to the Guide for the Care and Use of Laboratory Animals published by the National Institutes of Health (NIH publication No. 85-23, revised 1996). Studies on human volunteers were approved by the research ethics board of the Hospital for Sick Children, Toronto.

Induction of Remote Ischemic Conditioning (rIC) or Preconditioning (rIPC):

C57BL/6 male mice (8-10 weeks) were anesthetized with pentobarbital (60 mg/kg intraperitoneally). remote IC was induced by four cycles of 5 minutes of limb ischemia (by tourniquet) followed by 5 minutes reperfusion as previously described (Kharbanda et al. Circulation 2001, 103:1624; Konstantinov et al. J Thorac Cardiovasc Surg 2005, 130:1326).

MicroRNA Stem Loop RT-PCR:

Total RNA was extracted from left ventricular tissue using TRIzol Reagent (Invitrogen), according to the manufacturer's instructions. RT-PCR was performed using TagMan MicroRNA assay kit (ABI). Data were normalized by evaluating RNA U6 (RNU6B, ABI) expression.

Mouse Langendorff Preparation and Global Ischemia/Reperfusion Model:

In order to examine the myocardial effects of the interventions, without the potential confounding effects on other systems, isolated mouse hearts were mounted on the Langendorff perfusion apparatus as previously described (Kharbanda et al. Circulation 2001, 103:1624; Konstantinov et al. J Thorac Cardiovasc Surg 2005, 130:1326) and perfused under non-recirculating conditions at a constant pressure of 80 mmHg with 37° C. Krebs-Henseleit buffer (KHB). Then a balloon, made with saran wrap and PE60 polyethylene tubing, was inserted into left ventricular (LV) through the mitral valve and was connected to a pressure transducer. The balloon was inflated with water to adjust left ventricular end-diastolic pressure (LVEDP) to 7-10 mmHg at the beginning of the experiment and the volume kept constant for the duration of the study. After a 20 min stabilization period, hearts were subjected to 30 min of no-flow global ischemia followed by 60 min of reperfusion. Hemodynamic measurements, including heart rate (HR), peak left ventricular pressure (LVP), maximum rate of contraction (+dP/dtmax), maximum rate of relaxation (−dP/dtmin), and LVEDP will be recorded on a data acquisition system (PowerLab, ADInstruments) throughout the procedure.

Measurement of Infarct Size:

Infarct size was assessed via 1.25% 2,3,5-triphenyltetrazolium chloride (TTC, Sigma) staining as described previously (Kharbanda et al. Circulation 2001, 103:1624; Konstantinov et al. J Thorac Cardiovasc Surg 2005, 130:1326).

Antisense Oligonucleotide Preparation and Delivery:

Single-stranded RNAs were synthesized by VBC Biotech (Vienna), antagomiR-144: (5′-agUACAUCAUCUAUACugua-Chol-3′) (SEQ ID NO:3); and a scrambled (mutated) miRNA as a control (AntagomiR-Co/miR-Co: 5′-aaGGCAAGCUGACCCUGAaguu-Chol-3′) (SEQ ID NO:5). Each oligonucleotide was deprotected, desalted, and purified by high-performance liquid chromatography. Antagomir and control oligonucleotides were dissolved in PBS before administration. C57BL/6 mice received antagomiR-144, or antagomiR-Co (8 mg/kg body weight in 200 μl, per day) or a comparable volume of PBS (200 μl) through three consecutive daily tail vein injections. The dose was used based on an established protocol used by Bonauer et al. 2009 Science 324:1710.

Mice were divided into five groups as follows:

Group 1 (PBS, n=7), mice received three consecutive daily tail vein injections of PBS (200 μl). Hearts were isolated and mounted on Langendorff preparation (see below for expanded methods) for global ischemia/reperfusion experiments on the next day after final injection.

Group 2 (PBS+rIPC, n=6), similar to group 1 with additional rIPC performed prior to heart harvest.

Group 3 (AntimiR-Co+rIPC, n=5), mice received antagomir control (scrambled oligonucleotide) injection for 3 days with rIPC followed by ischemia/reperfusion performed on the next day after final injection.

Group 4 (AntimiR-144+rIPC, n=5) mice received 3 daily injections of antagomir-144 with rIPC followed by ischemia/reperfusion on the next day after final injection.

Group 5 (Antagomir-144 alone, n=5), mice received antagomir-144 alone followed by ischemia/reperfusion performed on the next day after final injection.

The following schematic illustrates effects of antagomir-144 on rIPC-induced cardioprotection in the mouse ischemia reperfusion model.

Preparation and Administration of miR-144:

Mature miR-144 was synthesized by VBC Biotech (Vienna), miR-144 sequences is: 5′-uaCAGUAUAGAUGAUGUAcuag-Chol-3′ (SEQ ID NO:4). miR-144 and control oligonucleotides were dissolved in PBS before administration. C57BL/6 mice received miR-144, or miR-Co (8 mg/kg body weight in 200 μl) or a comparable volume of PBS (200 μl) via tail vein injections. C57BL/6 mice were divided into four groups: Group 1 (PBS), mice received intravenous PBS and 60 minutes later, hearts were isolated and mounted on Langendorff preparation for global ischemia/reperfusion experiments. Group 2 (miR-Co), mice received miR-Co (200 μl, 8 mg/kg) followed by ischemia reperfusion; Group 3 (miR-144 Day1) mice received miR-144 (200 μl, 8 mg/kg), followed by ischemia/reperfusion after 50 minutes injection; Group 4 (miR-144 Day3), mice received miR-144 through three consecutive daily tail vein injections, global ischemia/reperfusion was performed on the next day after final injection.

The following figure illustrates the effect of systemic delivery of miR-144 on cardioprotection in the mouse ischemia reperfusion model (first and delayed windows of protection).

Immunoblotting:

Western blotting was conducted according to standard protocols. Phosphorylated-Akt (p-Akt) (Ser473) (cell signaling) and anti-CD63 (System Biosciences Inc.) was used as primary antibodies. Immunoblots were scanned using an Odyssey LI-COR and quantified using Image Studio.

Mouse and Human Plasma Preparation:

Remote IC was performed and blood was collected 15 minutes later in K2 EDTA tubes (Beckton Dickinson) and processed within 5 min for plasma preparation. Blood samples were first centrifuged at 1,500 g for 15 min at 4° C. The supernatant was collected and transferred to nuclease-free tubes, centrifuged again at 14,000 g for 15 min at 4° C. The supernatant was processed further for total RNA extraction. The human studies were approved by the institutional clinical research ethics board, and written informed consent was obtained from each subject. Blood samples were collected at pre (baseline) and post remote IC.

RNA Isolation:

A miRNeasy Mini Kit (Qiagen) was used to isolate total RNA from mouse and human plasma according to the manufacturer's instructions with cel-miR-39 (Qiagen) spiked for normalization of the RNA preparation.

Exosome Isolation and Measurement of Exosome Number by Nanoparticle Tracking Analysis:

Exosomes were isolated from mouse serum using ExoQuick (System Biosciences) according to the manufacturer's instructions. Exosome quantification and characterization of microparticles between 50-400 nm was performed using the NanoSight LM10-B system (NanoSight Ltd.).

Isolation of RNAs from Mouse Serum Exosomes and Exosome-Poor Supernatants:

Isolation of exosomal and supernatant RNAs was performed using the miRNeasy Mini Kit. Exosome or supernatant was diluted with 1 ml of QIAzol Solution according to the manufacturer's instructions with cel-miR-39 spiked for normalization of the RNA preparation. The levels of miR-144 were determined by MicroRNA Stem Loop RT-PCR, as described above. Precursor miR-144 level in mouse serum exosomes was measured using miScript Precursor Assays and miScript II RT Kit (Qiagen).

Argonaute-2 Co-Immunoprecipitation and RNA Extraction:

Using exosome-poor supernatant (250 uL), immunoprecipitation experiments were performed to determine whether miR144 co-fractionates with an Argonaute2 (Ago2) protein complex. 2 ug of Ago2 rabbit monoclonal antibody (Cell Signalling Inc.) or normal rabbit IgG antibody (Santa Cruz Inc.) was combined with 250 ul of exosome-free supernatant (prepared as described above). After overnight incubation at 4° C. to form immune complexes, the complexes were added to 20 μl of Resin Slurry (Pierce Classic IP kit) and incubated for 2 hours at 4° C. with constant shaking. The resin was then washed three times with cold IP lysis/wash buffer and the sample was eluted in 1 ml QIAzol and processed for RNA isolation.

Statistical Analysis:

Sample sizes for mouse remote IC and Langendorff experiment were chosen based on prior studies (Kharbanda et al. 2006, 92:1506). For all comparisons, statistical significance was determined using one way ANOVA, followed by post hoc testing (Newman-Keuls) where appropriate. Values of P<0.05 were considered statistically significant. Data are shown as mean±S.E. (standard error).

### Example 1

### miRNA Expression Profile in Remote IC Murine Hearts

### Methods:

MicroRNA Microarray Expression Profiling and Data Analysis.

Total RNA was extracted from left ventricular tissue using TRIzol Reagent (Invitrogen), according to the manufacturer's instructions. The Genetic and Genomic Biology facility at the Hospital for Sick Children determined miRNA expression using a mouse miRNA microarray containing 655 miRNA Beadchips (Illumina). RNA was amplified and subsequently hybridized to the SAM-Bead microarray, according to the instructions of the manufacturer, and BeadChips were subsequently scanned with the Illumina iScan Reader. Microarray data processing and analysis were done using Illumina BeadStudio software. The data was standardized by a quantile normalization method, and LIMMA (Linear Models for Microarray Data) analysis was used for statistical comparisons of control and remote IC or control and IPC profiles. The miRNAs selected had an adjusted p value <0.1 (false discovery rate (FDR) <0.1) and fold change >1.5 (up-regulated in rIPC group) or fold change <−1.5 (down-regulated in rIPC group).

MicroRNA Stem Loop Reverse Transcriptase Polymerase Chain Reaction (RT-PCR).

5 miRNAs (miR-27a*, miR-144, miR-489, miR-684, miR-141) were selected for validation by quantitative miRNA stem loop RT-PCR. cDNA was synthesized using TaqManRNA Reverse Transcriptase (Applied Biosystems (ABI)) according to manufacturer's instructions. RT-PCR was performed with the RT product using TaqMan MicroRNA assay kit (Applied Biosystems). Data were normalized by evaluating RNA U6 (RNU6B, ABI) expression.

### Results:

The results showed that 22 of 655 miRNAs were significantly modified by rIC (16 upregulated, 6 downregulated). Table 1 provides a list of miRNAs that are down-regulated by 1.5 fold or more (top panel) and up-regulated by 1.5 fold or more (bottom panel) in heart tissue following remote IC. As shown, miRNA-144 was up-regulated by about 1.5 fold relative to control. miR 451 was not significantly altered by rIC.

TABLE 1
miRNA differentially expressed in first window rIPC heart

| | | |
|---|---|---|
| miRNA ID | fold change | adjusted P value |

Down-regulated miRNAs >−1.5-fold

| | | |
|---|---|---|
| mmu-miR-682 | −2.786 | 0.005 |
| mmu-miR-712 | −2.126 | 0.043 |
| mmu-miR-27a* | −1.878 | 0.001 |
| mmu-miR-302d | −1.664 | 0.009 |
| mmu-miR-148a* | −1.543 | 0.026 |
| mmu-miR-203* | −1.533 | 0.043 |

Up-regulated miRNAs >1.5-fold

| | | |
|---|---|---|
| mmu-miR-689 | 2.169 | 0.075 |
| mmu-miR-323-3p | 1.863 | 0.023 |
| mmu-miR-142-5p | 1.719 | 0.011 |
| mmu-miR-19a | 1.706 | 0.015 |
| mmu-miR-341: 9.1 | 1.701 | 0.009 |
| mmu-miR-32 | 1.682 | 0.048 |
| mmu-miR-693-5p | 1.666 | 0.037 |
| mmu-miR-293* | 1.655 | 0.042 |
| mmu-miR-707 | 1.653 | 0.039 |
| mmu-miR-19b | 1.643 | 0.021 |
| mmu-miR-375 | 1.592 | 0.026 |
| mmu-miR-200c* | 1.521 | 0.043 |
| mmu-miR-96 | 1.520 | 0.015 |
| solexa-3062-153 | 1.517 | 0.023 |
| mmu-miR-144 | 1.542 | 0.011 |
| mmu-miR-489 | 1.500 | 0.005 |

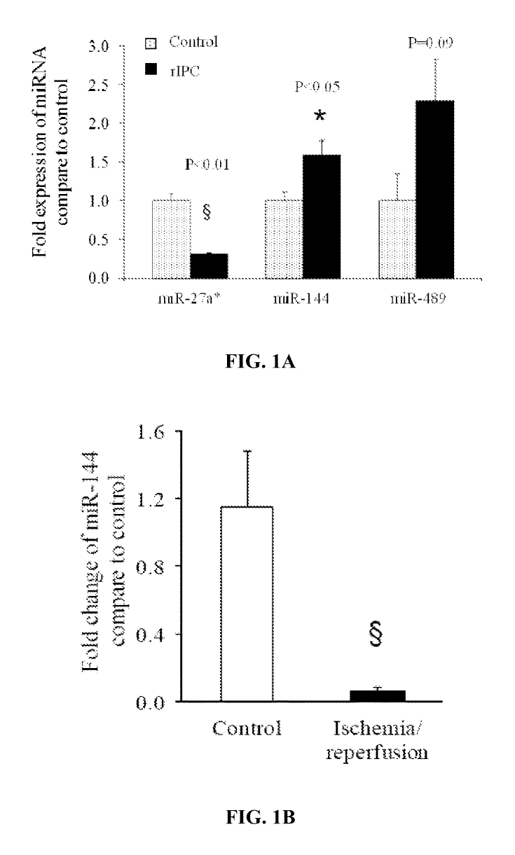

Four miRNAs (miR-144, miR-451, miR-27a-5p, miR-489) were selected for validation by quantitative miRNA stem loop RT-PCR. FIG. 1A shows the results of this validation study performed subsequent to the microarray analysis. The fold increase of miRNA-144 was confirmed using this independent assay. Furthermore, we found that IR injury alone led to a marked reduction in myocardial miR-144 levels. (FIG. 1B)

Consequently, the role of miR-144 in the cardioprotection induced by rIPC was tested by studying the effects of intravenous administration of antagomiR-144.

### Example 2

### Effects of Antagomir-144 on rIPC-Triggered Cardioprotection

### Methods:

Treatment of Mice with AntagomiR and mir144.

AntagomiRs were synthesized by VBC biotech (Vienna). Sequences are as follows:

AntagomiR-144: 5′-[agUACAUCAUCUAUACugua]-Chol-3′ (20 nts) (SEQ ID NO:3);
miR-144: 5′-[uaCAGUAUAGAUGAUGUAcuag]-Chol-3′ (22 nts) (SEQ ID NO:4); and
AntagomiR-Co: 5′-[aaGGCAAGCUGACCCUGAaguu]-Chol-3′ (22 nts) (SEQ ID NO:5).
Upper case letters represent phosphodiester (PO) bases;
Lower case letters represent phosphorothioate (PS) bonds;
Square brackets represent 2′-O-methyl-RNA; and
‘Choi’ represents linked cholesterol.

Each oligonucleotide comprised 2 phosphorothioate linkages at the 5′ end and 4 phosphorothioate linkages at the 3′ end, along with a cholesterol modification at the 3′ end.

AntagomiR oligonucleotides were deprotected, desalted, and purified by high-performance liquid chromatography.

C57/B6 mice (10 weeks old) received antagomiR-144, miR-144 or mutant antagomiR, or a comparable volume of PBS (200 mL) through three consecutive daily tail vein injections (3×10 mg/kg body weight). The levels of miRs (miR-144) were determined by qRT-PCR, as described above.

On next day after final injection (i.e., on day 4), remote ischemic conditioning followed by I/R (via a mouse Langendorff procedure) was performed.

### Results:

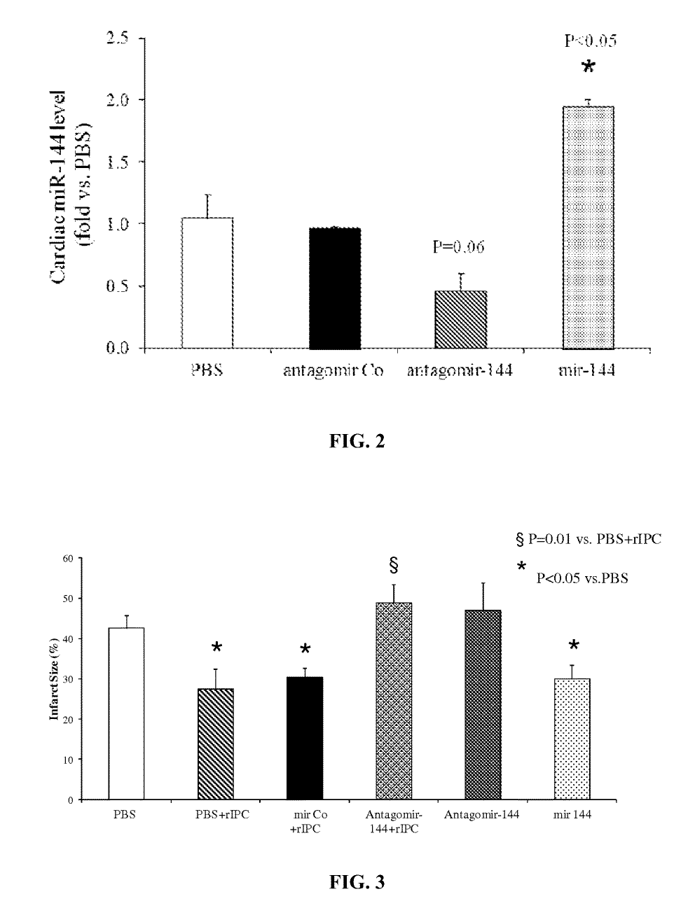

FIG. 2 shows the levels of cardiac miRNA-144 after tail vein injection of the antagomir-144 and negative and positive control oligonucleotides. The results show that antagomir-144 is able to reduce the level of miRNA-144 in vivo. miR-144 expression was reduced by 60% at 24 hours after the last of three daily intravenous injections of antagomiR-144. In contrast, the mutated antagomiR control had no effect on miR-144 expression level compared with the PBS treatment. These results indicated that injection of antagomir-144 efficiently decreases miR-144 levels in mouse heart.

In order to delineate the cardiac-specific effects of miR-144, we used a Langendorff isolated heart model of global IR injury. There were no statistically significant differences in baseline haemodynamic functional parameters among all groups (data not shown). Prior in vivo rIPC improved post-ischemic cardiac performance in isolated perfused hearts. On the basis of LVDP, recovery of post-ischemic contractile function was greater in PBS+rIPC, miR-Co+rIPC hearts than in PBS sham hearts. By the end of the 60 min reperfusion period, a significantly greater functional recovery was observed in PBS+rIPC (91.3±2.5% of pre-ischemic value), miR-Co+rIPC (94.3±3.2%) compared with PBS alone (77.7±1.3% of pre-ischemic value, p<0.01). These results indicated that post-ischemic contractile function was improved by rIPC. Diastolic recovery was also improved by rIPC, at the end of the reperfusion period, LVEDP was significantly lower in PBS+rIPC (15.9±2.5 mmHg), miR-Co+rIPC (16.2±1.1 mmHg) than in PBS treated hearts (24.2±2.7 mmHg, p=0.046 and p=0.037). In the group treated with antagomir-144 prior to rIPC (antagomir-144+rIPC), no significant differences in LVDP, or LVEDP, were seen relative to the PBS group.

Myocardial infarct size was assessed by TTC staining. Consistent with the improved functional recovery, infarct size was significantly reduced by PBS+rIPC (27.5±5%) and miR-Co+rIPC (30.4±2%) compared to PBS alone (44.7±3%, p=0.02 and p=0.004), and this effect was abrogated by injection of antagomir-144 (antagomir-144+rIPC: 49±4%, p=ns compared with PBS). Antagomir-144 injection alone did not result in a significant difference in infarct size compared to PBS treated hearts. These results indicate that antagomir-144 reverses rIPC-induced cardioprotection.

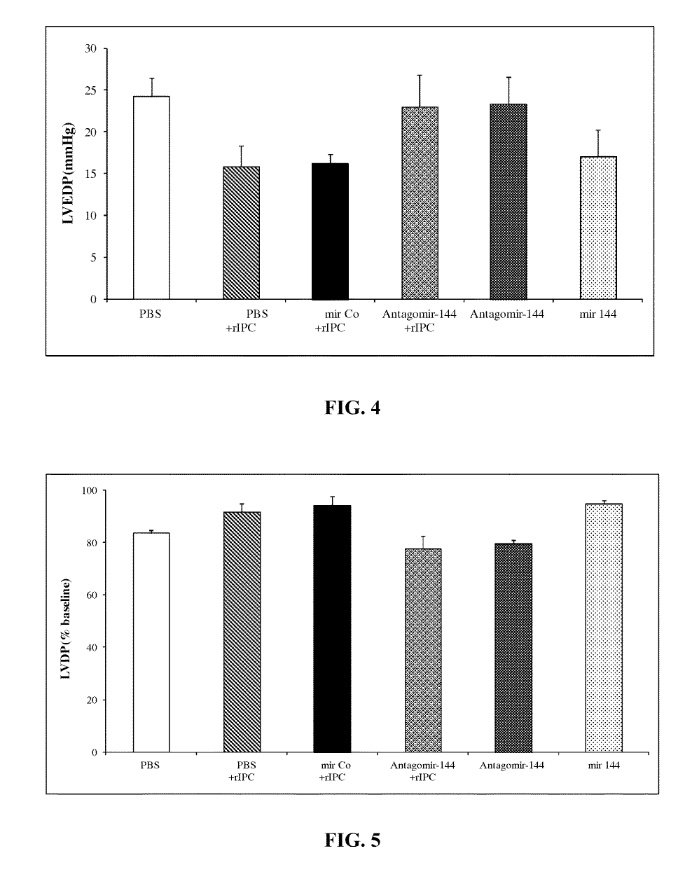

Similar data are provided in FIGS. 3-5. FIGS. 3-5 show the effects of remote IC alone, remote IC together with a control antagomir or with antagomir-144, antagomir-144 alone, and miRNA-144 alone on infarct size (FIG. 3), LVEDP (FIG. 4) and LVDP (FIG. 5). FIG. 3 shows that remote IC prior to the experimentally induced infarct results in a smaller infarct size. Administration of miRNA-144 is able to achieve a comparable benefit. Administration of antagomir-144 (but not a control antagomir) abrogates the benefit achieved using remote IC. Similar effects were seen with the hemodynamic functions shown in FIGS. 4 and 5. More specifically, on the basis of LVDP, recovery of post-ischemic contractile function was greater in PBS+rIPC, miR-Co+rIPC hearts than in PBS sham hearts (data not shown). By the end of the 60 minute reperfusion period, a significantly greater functional recovery was observed in PBS+rIPC (91.3±2.5% of pre-ischemic value), miR-Co+rIPC (94.3±3.2%) compared with PBS alone (77.7±1.3% of pre-ischemic value, p<0.01, data not shown). These results indicated that post-ischemic contractile function was improved by rIPC. Diastolic recovery was also improved by rIPC, at the end of the reperfusion period, LVEDP was significantly lower in PBS+rIPC (15.9±2.5 mmHg), miR-Co+rIPC (16.2±1.1 mmHg) than in PBS treated hearts (24.2±2.7 mmHg, p=0.046 and p=0.037, data not shown). In the group treated with antagomir-144 prior to rIPC (antagomir-144+rIPC), no significant differences in LVDP or LVEDP, were seen relative to the PBS group.

### Example 3

### miR-144 Level after rIPC

### Methods:

Sample Preparation.

Peripheral blood was collected into K2-EDTA tubes (Becton Dickinson, BD) and processed within 5 minutes for plasma preparation. Blood samples were first centrifuged at 1500 g for 15 minutes at 4° C. The supernatant was collected and transferred to nuclease-free tubes, centrifuged again at 14000 g for 15 minutes at 4° C. The supernatant was processed further for total RNA extraction or aliquoted and stored at −80° C.

Isolation of Total RNA from Mouse Plasma.

A miRNeasy Mini Kit (Qiagen) was used to isolate total RNA from human plasma, according to the manufacturer's instructions. Samples were spiked with cel-miR-39 (Qiagen) for normalization of the RNA preparation. The levels of miRs (miR-144) were determined by qRT-PCR, as described above.

### Results:

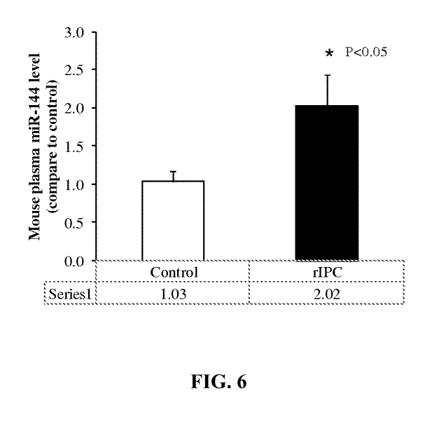

FIG. 6 shows that remote IC increases the level of plasma miRNA-144 compared to a control. The level of miRNA-144 was increased about 2 fold relative to control.

### Example 4

### Systemic Administration of miR-144 Induces Early and Delayed Cardioprotection in Mouse Ischemia Reperfusion Model

To examine if systemically delivered miR-144 can induce early or delayed cardioprotection, intravenous miR-144 was administered by tail vein injection and assessment of ischemia-reperfusion injury (Langendorff) was performed immediately after a single injection (miR-144 day1), or one day after three consecutive daily injections (miR-144 day3).

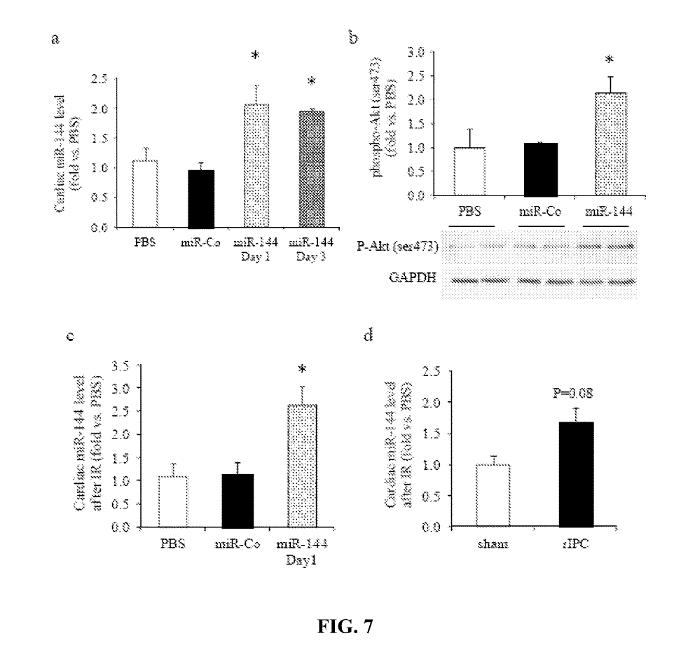

We found that miR-144 levels were increased over two-fold, compared to PBS control, both after 1 hour injection, and 1 day after three days of miR-144 injection (FIG. 7A). Furthermore, we showed increased levels of phosphorylated AKT (phospho-Akt), phosphor-GSK3b and phospho-p44/42 MAP Kinase in the myocardium one hour after miR-144 injection (FIG. 7B and data not shown), suggesting that mIR-144 recapitulates the early protective kinase response characteristic of the preconditioned phenotype (Li et al. 2011 Clin Sci 120:451). We then showed that the reduction in myocardial miR-144 levels (FIG. 1B) induced by IR injury was rescued both by pretreatment with intravenous miR-144 (FIG. 7C) and rIPC (FIG. 7D).

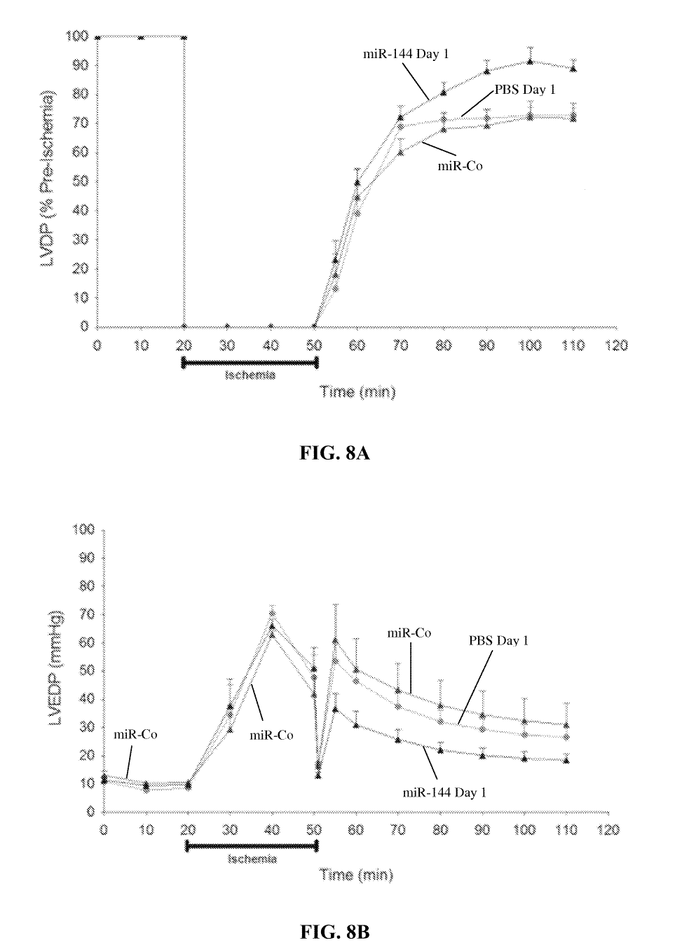

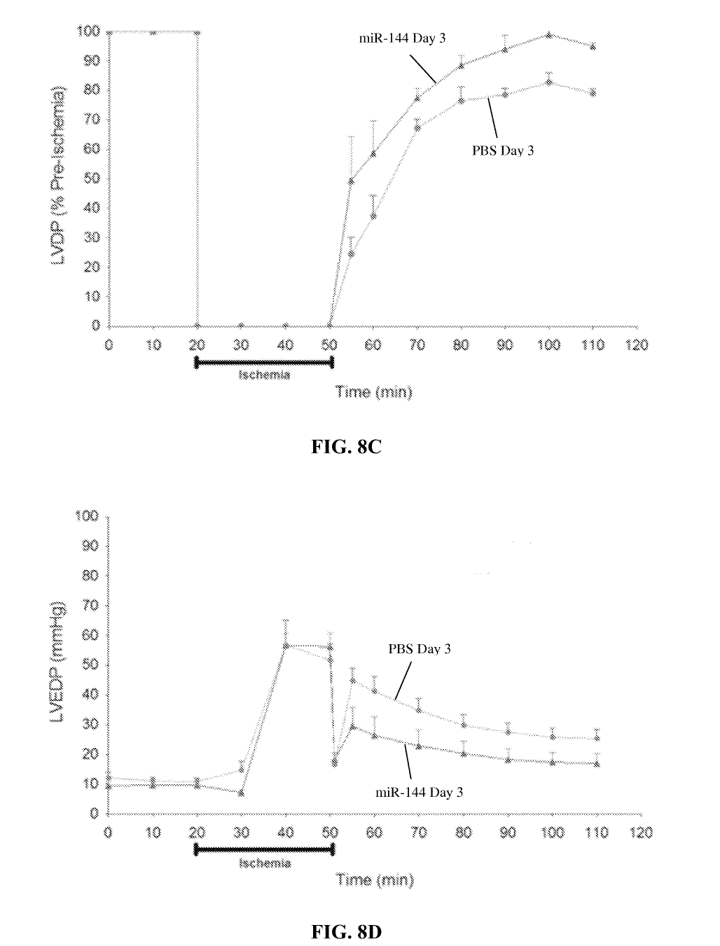

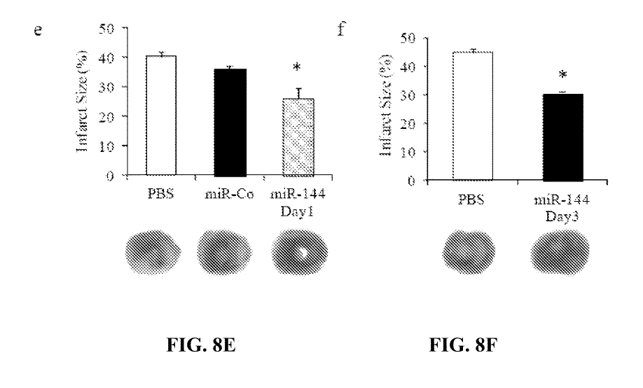

Hearts harvested from animals pre-treated with intravenous miR-144 or rIPC were equally protected against lethal IR injury at both 50 minutes (early window) and 24 hours (delayed window) after miR-144 treatment, as manifest by improved functional recovery (FIGS. 8A-D) and a significant reduction in infarct size (FIGS. 8E and 8F). By the end of the 60 min reperfusion period, a significantly greater functional recovery was observed in miR-144 Day1 (91.8±1.7%), compared with PBS alone (72.9±2.3% of pre-ischemic value, p<0.01, FIG. 4a) and miR-144 Day3 (94.9±1.2%), compared with PBS day3 (77.7±1.3% of pre-ischemic value, p<0.01, FIG. 8C). Diastolic recovery was also improved by miR-144, at the end of the reperfusion period, LVEDP was significantly lower in miR-144 Day1 (18.4±2.0 mmHg), miR-144 Day3 hearts (17.1±3.2 mmHg) than in PBS treated hearts (26.7±3.2 mmHg, 24.2±2.7 mmHg p=0.04 and p=0.079, FIGS. 4b and d). Infarct size was significantly reduced by miR-144 Day1 (25.9±4%) compared to PBS alone (39.0±2%, p=0.015, FIG. 8E). miR-144 Day3 (30.1±3%) compared to PBS Day3 (44.7±3%, p=0.012, FIG. 8F). These results confirm that miR-144 induces early and delayed cardioprotection.

### Example 5

### Circulating miR-144 after rIPC and Possible Mechanisms of Transport; rIPC Up-Regulates Circulating miR-144 Levels in Mouse and Humans

We next examined the effects of rIPC on circulating levels of miR-144. Fifteen minutes after completion of rIPC, there was an approximate 2-fold increase in plasma miR-144 levels in both rIPC-treated mice and human volunteers as shown in FIGS. 9A and B.

### Example 6

### Plasma Transport of miR-144

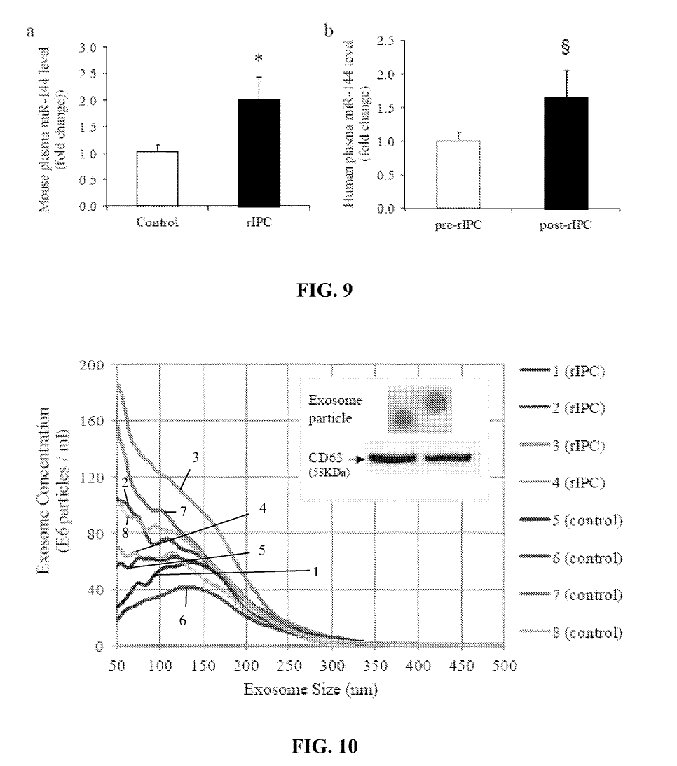

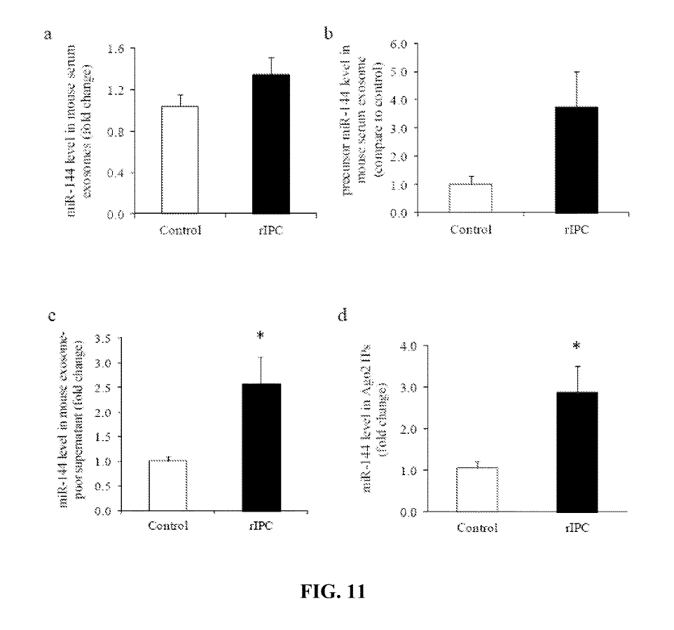

We next examined plasma microparticle (MP) responses to rIPC. Following separation and re-suspension, there was no difference in MP (50-400 nm) numbers as analysed by NanoSight (FIG. 10) and, using stem loop RT-PCR analysis, there was a non-significant increase in miR-144 (FIG. 11A). However, there was an almost 4-fold increase in miR-144 precursor (FIG. 11B) in the exosome pellet, and a significant increase in miR-144 levels in exosome-poor serum (FIG. 11C). To further investigate the binding of extracellular miRNA to Argonaute2 (Ago2), a known extracellular miRNA carrier, anti-Ago2 immunoprecipitates were subjected to TaqMan miRNA Assay. Our results show that Ago2-bound miR-144 levels increase following rIPC (FIG. 11D), suggesting that Ago2 also plays a role in extracellular miR-144 transport.

### DISCUSSION

This is the first study to examine the cardiac miRNA expression after remote ischemic conditioning (rIC). These results show that miR-144 plays a central role in the cardioprotection afforded by rIC. First we showed that rIC was associated with increased myocardial expression of miR-144 and that prior rIC and pre-treatment with intravenous miR-144 homologue oligonucleotide rescued the fall in miR-144 levels seen with ischemia-reperfusion (IR) injury. We then showed that rIC reduced infarct size and improved functional recovery in isolated hearts subjected to IR injury, and that these effects were completely abrogated by pretreatment of the donor animals with an anti-sense oligonucleotide against miR-144. Importantly, the effects of rIC were recapitulated by intravenous administration of miR-144 homologue oligonucleotide, there being an early window (associated with increased phosphor-AKT signal) within 50 minutes of administration and a delayed window of cardioprotection demonstrable 24 hours after 3 daily injections of miR-144.

Having subsequently demonstrated that plasma miR-144 levels are increased in mouse and humans subjected to rIC, we then examined the possible mechanisms of plasma transport of miR-144 as theoretically free micro-RNA in the plasma should be digested by plasma RNase. We showed that there was a 4-fold increase in hairpin miR-144 precursor in the exosomal fraction, and a marked increase in miR-144 levels in the exosome-free plasma supernatant, suggesting an alternate plasma carriage mechanism. We therefore examined a relatively recently described mechanism of miR transport, carriage within complexes of the protein Argonaute-2 (Ago-2) (Chen et al. 2012, Protein Cell, 3:28-37). In subsequent experiments, we were able to demonstrate co-immunoprecipitation of plasma miR-144 and Ago-2 suggesting this as the mechanism of plasma transport of miR-144 after rIC, and possibly after intravenous administration. These novel observations establish a pivotal role for miR-144 in the cardioprotection associated with rIC, and suggest miR-144 as a potential cardioprotective therapy.

### Other Embodiments

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-18. (canceled)

19. A method of treating a subject at risk of experiencing, or a subject experiencing, or a subject that has experienced an ischemic injury comprising administering to the subject in need thereof an miRNA-144 therapy in an effective amount.

20. (canceled)

21. The method of claim 19, wherein the ischemic injury is a myocardial infarction, a stroke, restenosis, or traumatic injury.

22. The method of claim 19, wherein the subject is in need of cardioprotection and the miRNA-144 is administered before, during and/or following an ischemic event that causes cardiac ischemia.

23. (canceled)

24. The method of claim 19, wherein the subject is not at risk of experiencing, or is not experiencing, or has not experienced a myocardial infarction or other event that causes cardiac ischemic injury.

25. The method of claim 19, wherein the ischemic injury is surgery.

26. The method of claim 25, wherein the surgery is cardiac surgery or cardiovascular surgery.

27. The method of claim 19, wherein IC or an IC-like interventions has been, is being, and/or will be performed on the subject.

28. The method of claim 27, wherein the IC-like intervention is transcutaneous electrical nerve stimulation.

29. The method of claim 19, wherein the subject is a human.

30. The method of claim 19, further comprising administering to the subject an additional therapeutic agent.

31. The method of claim 19, further comprising administering to the subject an angiotensin-converting enzyme (ACE) inhibitor.

32. The method of claim 19, further comprising administering to the subject an angiotensin II receptor blocker.

33. The method of claim 19, further comprising administering to the subject an anti-platelet therapy.

34-37. (canceled)

38. The method of claim 19, wherein the miRNA-144 therapy comprises a nucleic acid consisting of a nucleotide sequence of SEQ ID NO: 1, 2, 4 or 6.

39. The method of claims 19-38, wherein the miRNA-144 therapy comprises nucleic acid comprising a nucleotide sequence of SEQ ID NO: 1, 2, 4 or 6 or that is complementary to SEQ ID NOs: 1, 2, 4 or 6.

40. The method of 39, wherein the nucleic acid comprises one or more non-naturally occurring backbone linkages, one or more non-naturally occurring nucleotide or nucleotide analogs or nucleotide modifications.

41. (canceled)

42. The method of claim 40, wherein the nucleic acid comprises a cholesterol modified nucleotide or nucleotide analog.

43. A method for enhancing physical performance comprising administering an miRNA-144 therapy to a subject prior to a physical activity in order to enhance performance of the physical activity by the subject.

44-48. (canceled)

49. The method of claim 43, wherein the miRNA-144 therapy comprises nucleic acid comprising a nucleotide sequence of SEQ ID NO:
1, 2, 4 or 6 or that is complementary to SEQ ID NOs: 1, 2, 4 or 6.

50-52. (canceled)


SEQUENCE LISTING (7 sequences)

SEQ ID NO: 1 - RNA, 86 nt, Homo sapiens
uggggcccuggcugggauaucaucauauacuguaaguuugcgaugagacacuacaguauagaugauguacuaguccgggcaccccc

SEQ ID NO: 2 - RNA, 20 nt, Homo sapiens
uacaguauagaugauguacu

SEQ ID NO: 3 - RNA, 20 nt, Artificial Sequence
  AntagomiR-144
aguacaucaucuauacugun

SEQ ID NO: 4 - RNA, 22 nt, Artificial Sequence
  miR-144
uacaguauagaugauguacuan

SEQ ID NO: 5 - RNA, 22 nt, Artificial Sequence
  AntagomiR-Co
aaggcaagcugacccugaagun

SEQ ID NO: 6 - RNA, 22 nt, Artificial Sequence
  Synthetic Polynucleotide
uacaguauagaugauguacuag

SEQ ID NO: 7 - RNA, 23 nt, Homo sapiens
aaaccguuaccauuacugaguuu